SEQUENCE LISTING (10 sequences)

SEQ ID NO: 1 - protein, 5 aa, Human coronavirus (HCoV-OC43)
NDKDT

SEQ ID NO: 2 - protein, 16 aa, Human coronavirus (HCoV-OC43)
LKGSVLLSRLWFKPPF

SEQ ID NO: 3 - protein, 6 aa, Human coronavirus (SARS-CoV-1)
CTTFDD

SEQ ID NO: 4 - protein, 8 aa, Human coronavirus (SARS-CoV-1)
INHTFGNP

SEQ ID NO: 5 - protein, 11 aa, Avian coronavirus (IBV)
YYYQSAFRPPN

SEQ ID NO: 6 - protein, 6 aa, Human coronavirus (MERS-Cov)
QQTFFD

SEQ ID NO: 7 - protein, 19 aa, Human coronavirus (MERS-CoV)
GHATGTTPQKLFVANYSQD

SEQ ID NO: 8 - protein, 4 aa, Human coronavirus (SARS-CoV-2)
TTRT

SEQ ID NO: 9 - protein, 15 aa, Human coronavirus (SARS-CoV-2)
IHVSGTNGTKRFDNP

SEQ ID NO: 10 - protein, 300 aa, Artificial Sequence
  Primary sequence of truncated SARS-COV-2 (Lineage A)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETK

(19) United States
(12) Patent Application Publication
(10) Pub. No.: US 2021/0387187 A1
(43) Pub. Date: Dec. 16, 2021

(54) FLOW DEVICE

(71) Applicant: University of Warwick, Coventry Warwickshire (GB)

(72) Inventors: Matthew Ian Gibson, Coventry (GB); Sarah-Jane RICHARDS, Coventry (GB); Alexander Neil BAKER, Leamington Spa (GB)

(21) Appl. No.: 17/330,986

(22) Filed: May 26, 2021

(30) Foreign Application Priority Data
May 27, 2020 (GB) 2007895.2
Feb. 16, 2021 (GB) 2102159.7

(51) Int. Cl. B01L 3/00; G01N 33/543; G01N 33/58; G01N 33/569

(52) U.S. Cl. CPC B01L 3/502715; B01L 3/50273; B82Y 5/00; G01N 33/587; G01N 33/56983; G01N 33/54353

(57) ABSTRACT

The present invention relates to methods for the detection of an analyte, such as coronavirus (e.g. SARS-CoV-2) or other virus particles and proteins, in a test sample. The invention also provides a flow device for use in such methods. Additionally, there is provided a coronavirus-binding reagent having the structure [sialic acid]-[linker]-[polymer]-[gold nanoparticle] for use in the devices and methods of the invention.

The present invention relates to methods for the detection of an analyte, such as coronavirus (e.g. SARS-CoV-2) or other virus particles and proteins, in a test sample. The invention also provides a flow device for use in such methods. Additionally, there is provided a coronavirus-binding reagent having the structure [sialic acid]-[linker]-[polymer]-[gold nanoparticle] for use in the devices and methods of the invention.

In December 2019 a novel zoonotic coronavirus (SARS-COV-2) was discovered in Wuhan, China. This virus has triggered a pandemic and it is the causative agent of the respiratory disease COVID-19.¹ There are currently no approved therapeutic treatments against this virus, nor a vaccine. Diagnostics, surveillance and case isolation are therefore the primary tools for controlling its spread in a population to drive down the basic reproduction (R₀) value. Following genome sequencing of the novel coronavirus, RT-PCR (reverse transcription polymerase chain reaction) based diagnostics were rapidly established. RT-PCR requires dedicated laboratory facilities and trained personal, and does not provide an instant output. While RT-PCR is highly specific, false negatives are possible: Xie et al. reported 3% false negatives versus chest CT scans for COVID-19 patients;² there are also reports of conflicting RT-PCR results in samples from the same patient.^{3,4} Additionally, the sampling location, i.e. throat versus lower respiratory tract, can impact on the rate of false negatives.⁵

Alternative detection platforms to RT-PCR include lateral flow devices (LFDs) and flow-though devices, typically using antibodies as the detection units, with the most famous being the home-pregnancy test.⁶ In such devices, an antibody is immobilized to both the stationary phase (e.g. nitrocellulose paper) and also to the mobile phase (e.g. gold nanoparticles), forming a ‘sandwich’ with the antigen, and hence test lines show a positive (e.g. red line) response. As they are paper-based, they are also extremely low cost. The cost-effectiveness of point-of-care lateral flow systems are well demonstrated by various studies of malaria rapid-diagnostic tests^{7,8} and they were found to compare well against the more expensive RT-PCR for Ebola-diagnostic devices.⁹

In addition to antibodies, other biological molecules such as nucleic acids¹⁰ and lectins¹¹ have also been used in diagnostic devices. Glycans have not been widely used in lateral flow devices however, but offer opportunities beyond antibodies, particular in terms of stability, as they do not require a cold-chain and can tolerate variations in heat and humidity. They are therefore ideal for low-resource, triage or emergency settings.

In vivo, glycans (carbohydrates) direct a myriad of binding and recognition events from cell-cell communication to markers of disease. Analysis of influenza zoonosis (species crossing), which lead to the swine flu pandemic of 2009, showed that viral hemagglutinins which normally bind to 2,3-sialic acids in respiratory tracts switched to a human disease by binding to 2,6-sialic acids instead.¹² This switch in glycan affinity has allowed biosensors to be established to identify rapidly which strain is present without the need for genome sequencing or PCR-based methods.^{13,14}

All coronaviruses display homotrimers of spike glycoproteins on their surface. Sialic acid binding by the S1 spike protein subunits has been shown to be crucial for coronaviruses to engage host cells, whilst the S2 domain initiates virus-cell fusion.¹⁵ Tortorici et al. showed the structural basis for 9-O-acetylated sialic acid binding to a human coronavirus (strain OC43) by Cryo-EM; affinity to this ligand by the HKU1-HE strain has also been found.^{16,17} MERS S1 preferentially binds 2,3- over 2,6-linked sialic acids, but acetylation decreases affinity.¹⁸

However, reports on the binding of the SARS-CoV-2 spike protein to glycans have indicated that the SARS-CoV-2 spike protein does not bind to sialic acid residues (Hao et al., (2020) bioRxiv preprint doi: https://doi.org/10.1101/2020.05.17.100537).

The glycobiology of coronaviruses have not yet been explored in detail. However, the inventors have recognised that the above examples demonstrate that glycan binding function is conserved across many strains, and, due to its role in ‘anchoring’ the virus, this offers opportunities for detection of the virus using capture techniques such as LFD.

In direct contrast to the above, the inventors have now demonstrated that a sialic acid-based lateral flow detection system can be used to recognize the spike glycoprotein from the SARS-CoV-2 virus, the causative agent of the COVID-19 pandemic.

Sequence alignments within previous coronaviruses showed little homology between sialic acid binding sites. However, polymer tethers were used by the inventors to immobilize 2-amino-2-deoxy-N-acetylneuraminic acid onto gold nanoparticles to give signal-generating components, present in the essential format for flow devices. Against the teachings of Hao et al., biolayer interferometry showed strong affinity of these particles for the SARS-CoV-2 spike protein. Lateral flow (paper-based) assays showed that the nanoparticles could detect SARS-CoV-2 spike protein and that this was selective compared to the spike protein from SARS-CoV-1; it also had low affinity to intact deactivated influenza virus.

This represents a key step forward in developing low cost diagnostics suitable for point-of-care, or even point-of-work/travel, to enable surveillance of this pandemic virus, without requiring any infrastructure and minimal training.

The invention may also be used for the detection of other viruses.

It is an object of the invention therefore to provide a method for the detection of an analyte, such as coronavirus (e.g. SARS-CoV-2) or other virus particles and proteins, in a test sample. It is further object of the invention to provide a flow device, e.g. a lateral flow device or flow-through device, for use in such methods. Additionally, there is provided a coronavirus- or other virus-binding reagent for use in the devices and methods of the invention, preferably having the structure [sialic acid]-[linker]-[polymer]-[gold nanoparticle].

In one embodiment, the invention provides a method of determining the presence of coronavirus particles or coronavirus proteins in a test sample, the method comprising the steps:
(a) contacting the test sample with a first specific binding partner, wherein the first specific binding partner comprises a terminal sialic acid, and wherein the first specific binding partner is linked to a detectable label; and
(b) detecting the presence or absence of detectable label which is bound to the test sample,
   wherein the presence of detectable label which is bound to the test sample is indicative of the presence of a coronavirus particle or coronavirus protein in the test sample.

In some embodiments, the test sample is first immobilised on a solid support. In other embodiments, the first specific binding partner comprises a solid support (e.g. particle or bead).

In some preferred embodiments, the method comprises the steps:
(a) immobilising the test sample in a detection zone on a solid support;
(b) contacting the solid support with a first specific binding partner, wherein the first specific binding partner comprises a sialic acid, and wherein the first specific binding partner is linked to a detectable label; and
(c) detecting the presence or absence of bound label in the detection zone,
   wherein the presence of bound label in the detection zone is indicative of the presence of a coronavirus particle or coronavirus protein in the test sample.

Preferably, the coronavirus is SARS-CoV-2. Preferably, the protein is a spike protein, more preferably a S1 spike protein. Preferably, the sialic acid is a terminal sialic acid.

In another embodiment, the invention provides a method of determining the presence of an analyte in a test sample, the analyte comprising coronavirus particles or coronavirus spike proteins, the method comprising the steps:
(a) contacting a flow device (preferably a lateral flow device or a flow-though device) comprising a conjugate zone and a detection zone with the test sample, wherein
   (i) the conjugate zone comprises a first specific binding partner wherein the first specific binding partner is linked to a detectable label, and wherein the first specific binding partner is not immobilised in the conjugate zone; and
   (ii) the detection zone comprises a second specific binding partner, wherein the second specific binding partner is immobilised in the detection zone,
wherein the first specific binding partner and/or the second specific binding partner comprise a sialic acid, and wherein the first or second specific binding partners which do not comprise a sialic acid comprise a ligand which binds to the analyte; and
(b) detecting the presence or absence of bound label in the detection zone,
   wherein the presence of bound label in the detection zone is indicative of the presence of the analyte in the test sample.

An appropriate aqueous solution is used to transfer the first specific binding partner to the detection zone, e.g. from a sample receiving zone.

Preferably, the sialic acid is a terminal sialic acid.

In another embodiment, the invention provides a flow device (preferably a lateral flow device or a flow-though device) for detecting the presence of an analyte in a test sample, the device comprising a conjugate zone and a detection zone, wherein:
(a) the conjugate zone comprises a first specific binding partner for the analyte, wherein the first specific binding partner is linked to a detectable label, and wherein the first specific binding partner is not immobilised in the conjugate zone; and
(b) the detection zone comprises a second specific binding partner for the analyte, and wherein the second specific binding partner is immobilised in the detection zone,
   characterised in that the first specific binding partner and/or the second specific binding partner comprise a sialic acid.

Preferably, the analyte is a virus particle or a virus surface protein, e.g. sialic acid binding virus, more preferably a coronavirus particle or a coronavirus spike protein, and most preferably a SARS-CoV-2 virus particle or a SARS-CoV-2 S1 protein. Preferably, the sialic acid is a terminal sialic acid. In some embodiments, the virus is an influenza virus, e.g. H3.

In another embodiment, the invention provides a compound having the structure a sialic acid-linker-polymer-gold nanoparticle

wherein the terms “sialic acid”, “linker” and “polymer” are as defined herein.

In one embodiment, the invention provides a flow device (preferably a lateral flow device or a flow-though device) and uses thereof for detecting the presence of an analyte in a test sample. Although the invention is exemplified herein with reference to lateral flow devices, the invention should not be seen as being limited in this way. Lateral flow devices (LFDs) and flow-though devices are often used to test a liquid sample, such as saliva, blood or urine, for the presence of an analyte. Examples of lateral flow devices include home pregnancy tests, home ovulation tests, tests for other hormones, tests for specific pathogens and tests for specific drugs. For example, EP 0291194 describes a lateral flow device for performing a pregnancy test.

The features of lateral flow devices and flow-though devices are well known in the art. Reference may be made, for example, to the following which describe general features of lateral flow devices, including methods of their production, and methods of linking detectable labels and immobilising reagents: EP2453242, US2015176050, WO 2020/049444, US 2020/0023354 A1, JP 2019023647 A, EP 0291194 A1, WO 2020/033235 A1, WO2019122816 (A1), WO 2019/023597, US 2020132693 A1, WO 2020/041267 A2, US 2018/372733 (A1), US 2018/133343 (A1), US2016017065 (A1), the contents of which are all specifically incorporated herein by reference.

Flow devices generally include the following discrete zones (a)-(c), and optionally (d) and (e), which are in fluid communication with one another, optionally in this order.

(a) A sample receiving zone. This zone receives the test sample comprising the analyte to be tested for.

The liquid sample is generally drawn by capillary action (or “wicking”) to the next zone. In some embodiments, the sample is transported by active fluid flow from the sample receiving zone to the subsequent zones.

(b) A conjugate zone. This zone comprises first specific binding partners for the analyte. The first specific binding partner is linked to a detectable label. The first specific binding partners are not immobilised in the conjugate zone; they are capable of being mobilised, i.e. being transported to subsequent zones by capillary action or active fluid flow.

The labelled first specific binding partners are retained (generally in dry form) in the conjugate zone prior to use, but will be free to migrate with the liquid sample (which leads to their reconstitution or activation). For example, in LFDs which are based on a porous material substrate, the test sample will be taken up in the sample receiving zone and then drawn through the porous material to the conjugate zone. When the porous material of the conjugate zone is moistened, the labelled first specific binding partners will be free to bind to the analyte (if present) and they are then transported to the detection zone.

Hence, in the conjugate zone, the first specific binding partners will bind to the analyte, if any analyte is present in the test sample. The liquid sample is then drawn by capillary action or active fluid flow to the next zone.

(c) A detection zone. This zone comprises a second specific binding partner for the analyte. The second specific binding partner is immobilised, i.e. it cannot be mobilised by the action of the liquid test sample. Generally, the second specific binding partner is not linked to a detectable label. The second specific binding partner may comprise the same or different analyte-binding moieties as the first specific binding partner.

The binding partners may participate in either a “sandwich” or a “competition” assay.

(d) Optionally, the flow device (preferably a lateral flow device or a flow-though device) may comprise a control zone, which provides a positive or negative control for the binding reaction.

(e) Optionally, the flow device (preferably a lateral flow device or a flow-though device) may comprise an absorbent zone. This acts as a sink for the liquid sample.

In this way, the test sample progresses from the sample receiving zone, through the conjugate zone and into the detection zone, and optionally through the control zone and/or to the absorbent zone.

Thus in one embodiment, the LFD comprises:
(a) a sample receiving zone, to which the test sample is applied or is capable of being applied;
(b) a conjugate zone, wherein the first specific binding partner is linked to a detectable label, and wherein the first specific binding partner is not immobilised;
(c) a detection zone, wherein the second specific binding partner is immobilised in the detection zone;
   and optionally one or both of:
(d) a control zone, and
(e) an absorbent zone,
   wherein the above zones, when present, are joined in (fluid) communication, in the above-mentioned order.

In some embodiments a second specific binding partner is not used.

In some embodiments, the sample is applied directly onto the detection zone, and immobilised there.

The invention also provides a method of determining the presence of an analyte in a test sample, the analyte comprising virus particles or proteins, e.g. coronavirus particles or coronavirus spike proteins, the method comprising the steps:

(a) contacting a flow device (preferably a lateral flow device or a flow-though device) comprising a conjugate zone and a detection zone with the test sample, wherein
(i) the conjugate zone comprises a first specific binding partner, wherein the first specific binding partner comprise a sialic acid linked to a detectable label, and wherein the first specific binding partner is not immobilised in the conjugate zone; and
(ii) the test sample is applied to the detection zone,
   (b) detecting the presence or absence of bound label in the detection zone,
   wherein the presence of bound label in the detection zone is indicative of the presence of the analyte in the test sample.

An appropriate aqueous solution is used to transfer the first specific binding partner to the detection zone, e.g. from a fluid receiving zone.

The invention also provides a flow device (preferably a lateral flow device or a flow-though device) for detecting the presence of an analyte in a test sample, the device comprising a conjugate zone and a detection zone, wherein:

(a) the conjugate zone comprises a first specific binding partner for the analyte, wherein the first specific binding partner comprises a sialic acid linked to a detectable label, and wherein the first specific binding partner is not immobilised in the conjugate zone; and
(b) the detection zone comprises a zone which is adapted to receive the test sample.

In some embodiments, the flow device (preferably a lateral flow device or a flow-though device) comprises:

(a) a fluid receiving zone, to which an aqueous solution is applied or is capable of being applied;
(b) a conjugate zone, wherein the first specific binding partner is linked to a detectable label, and wherein the first specific binding partner is not immobilised;
(c) a detection zone, to which the test sample is applied or is capable of being applied;
and optionally one or both of:
(d) a control zone, and
(e) an absorbent zone,
wherein the above zones, when present, are joined in (fluid) communication, in the above-mentioned order.

In these embodiments, the test sample is applied directly onto the detection zone. An aqueous solution (e.g. a pharmaceutically-acceptable diluent, carrier or excipient, or silver stain solution) is then applied to the fluid receiving zone. This fluid progresses through the conjugate zone (carrying the first specific binding partner) and into the detection zone, and optionally through to the control zone and/or to the absorbent zone.

In this embodiment, the first specific binding partner preferably comprises a sialic acid as defined herein, preferably conjugated to a nanoparticle as defined herein.

In one embodiment, the LFD may comprise a porous planar substrate or solid support comprising one or more discrete zones as defined herein. In one simple form, the LFD comprises a porous strip or chromatographic strip comprising a one or more discrete zones (as defined herein), along which the liquid test sample may be drawn by capillary action.

The strip may, for example, be paper, nitrocellulose, polyvinylidene fluoride, nylon or polyethersulfone. The use of such strips is well known in the art.

In other embodiments, the device (preferably a lateral flow device or a flow-though device) comprises one or more flow paths or channels in fluid communication with and between one or more discrete zones (e.g. (a)-(c) as described above). The device may be a microfluidic device. It may additionally comprise a pump, i.e. to move the fluids between the zones.

A typical LFD comprises a hollow casing constructed of moisture-impervious solid material (which may be opaque or transparent, but will generally include visually-readable portions at detection and control Zones) containing a dry porous carrier which communicates directly or indirectly with the exterior of the casing such that a liquid test sample can be applied to the porous carrier at the sample receiving zone and be transported to the other zones.

The test sample will be in liquid form, preferable an aqueous liquid or may be capable of being rehydrated. The test sample will generally comprise one or more biological samples from the subject.

The biological sample may be a bodily fluid from the subject, e.g. saliva, blood, plasma, serum, sweat, sputum, lacrimal fluid, urine, nasal swab or wash, throat swab or wash, or mouth swab or wash. The biological sample may also be waste water (e.g. to monitor the spread of disease). The biological sample may comprise cells, e.g. cells obtained from swabbing a part of the subject. The biological sample may also comprise a tissue biopsy, e.g. of tissue from the mouth, throat, trachea, bronchi or lungs. The biological sample may also comprise faecal tissue.

Preferably, cells and other solid materials are removed from the test sample before application to the sample receiving zone (e.g. by lysis and/or centrifugation). Any cells which are present in the biological sample should preferably be lysed and cell membranes removed before application to the sample receiving zone.

More preferably, the biological sample comprises material obtained from a nasal swab or throat swab from the subject or sputum from the subject.

The test sample may additionally comprise a pharmaceutically-acceptable diluent, carrier or excipient.

The test sample may also comprise suitable amounts and concentrations of buffers, salts, surfactants and/or blocking agents. These may be used to enhance the sensitivity and/or specificity of the methods. Blocking agents may include polymers, proteins and polysaccharides. Polymers include polyvinyl pyrrolidone, poly(vinylalcohol) and poly(ethylene glycol). Proteins include BSA (bovine serum albumin) and casein. Polysaccharides include those from milk powder.

The subject is preferably a mammalian subject. The mammal may be human or non-human. For example, the subject may be a farm mammal (e.g. sheep, horse, pig, cow or goat), a companion mammal (e.g. cat, dog or rabbit) or a laboratory test mammal (e.g. mouse, rat or monkey).

Preferably, the subject is a human. The subject may be male or female. The subject may be alive or dead (e.g. for post-mortem studies).

The human may, for example, be 0-10, 10-20, 20-30, 30-40, 40-50, 50-60, 60-70, 70-80, 80-90, 90-100 or above 100 years old.

The human may be one who is suffering from or at risk from a particular disease or disorder, e.g. SARS-CoV-2 or influenza. In other embodiments, the human is one who is suffering from Type 1 or Type 2 diabetes; one who has a heart disorder; or one who has chronic kidney disease.

Preferably, the analyte is a virus particle or a viral surface protein, or a derivative which is obtainable or obtained therefrom. In some embodiments, the virus is a virus which is capable of binding to sialic acid. In some embodiments, the virus is a respiratory virus, e.g. influenza.

More preferably, the analyte is a coronavirus particle or a coronavirus surface protein. The coronavirus may, for example, be severe acute respiratory syndrome (SARS), such as SARS-CoV-1 or SARS-CoV-2, or Middle East respiratory syndrome (MERS). Most preferably, the analyte is a SARS-CoV-2 particle or a SARS-CoV-2 surface protein.

Preferably, the surface protein is the spike protein, more preferably the S1 spike protein. In a particularly preferred embodiment, the analyte is a SARS-CoV-2 particle or the analyte is or comprises a SARS-CoV-2 S1 spike protein. In some embodiments, the analyte is an influenza virus, e.g. H3.

As used herein, the term “particle” includes particles which have been chemical-, heat- or radiation-treated, and particles which have been chemical-, heat- or radiation inactivated.

In practical terms, the biological sample (e.g. nasal or throat swab) will be obtained from the subject, and the cells obtained will be suspended in a physiologically-acceptable medium (e.g. water or PBS).

Any virus particles within the cells will be released from the cells by permeabilising the cells with an appropriate detergent, and then the cells and viruses will be separated from one another my centrifugation, leaving an aqueous suspension of the virus particles.

The virus particles may be chemical-, heat- or radiation inactivated. Alternatively, the virus particles may not have been inactivated (i.e. the virus particles are ones which are not chemical-, heat- or radiation inactivated).

This particle or derivatives thereof may then be tested (as the analyte) in a device of the invention. In other embodiments, the biological sample from the subject may be tested (as the analyte) without pre-treatment.

In the methods and devices of the invention, the first specific binding partner and/or the second specific binding partner comprise a sialic acid.

In some embodiments, the first specific binding partner and/or the second specific binding partner consist of or comprise a sialic acid of formula:

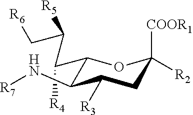

wherein

R1=H or a metal ion (M⁺);

R2=O-alkyl, 0-glycosyl, N-alkyl, triazole, S-alkyl or S-glycosyl;

R3=H, OH, NHAc, F, NH₂, N₃, triazole, O-alkyl or O-acetyl, N-glycolyl, N-acetamide or sulphonamide;

R4=H, OH, NHAc, F, NH₂, N₃, triazole, O-alkyl or O-acetyl, N-acetamide or sulphonamide;

R5=H, OH, NHAc, F, NH₂, N₃, triazole, O-alkyl or O-acetyl, N-acetamide or sulphonamide;

R6=H, OH, NHAc, F, NH₂, N₃, triazole, O-alkyl or O-acetyl, O-phosphate, N-acetamide or sulphonamide;

R7=NHAc, OH, NH₂, F, N₃, triazole, O-alkyl or O-acetyl, N-alkyl, N-glycolyl, N-acetamide or sulphonamide;

wherein one of R1-R7 (preferably R2) may be the point of attachment to a linker or a polymer,
and tautomers, enantiomers and diastereomers thereof.

Preferably, the first specific binding partner and/or the second specific binding partner consist of or comprise a sialic acid of formula:

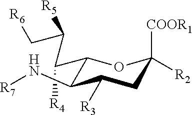

wherein

R1=H, acetyl, methyl or ethyl or a metal ion (M⁺),

R2=H, OH, O-alkyl, NH₂, N-alkyl, triazole or S-alkyl,

R3=H, OH, O-alkyl or O-acetyl,

R4=H, OH, O-alkyl or O-acetyl,

R5=H, OH, O-alkyl or O-acetyl,

R6=H, OH, O-alkyl or O-acetyl, and

R7=H or C(O)-alkyl or N-alkyl,

wherein one of R1-R7 (preferably R2) may be the point of attachment to a linker or a polymer,
and tautomers, enantiomers and diastereomers thereof.

As used herein, the term “alkyl” includes C₁₋₆ linear or branched alkyl chains, e.g. methyl, ethyl, propyl, butyl, pentyl and hexyl. The metal ion may be any monovalent ion, e.g. Na⁺. In some embodiments, one or more of the H groups within the alkyl group may independently be replaced by halogen, e.g. Cl or F.

As used herein, the term “glycosyl” includes a monosaccharide (e.g. galactose, glucose), a disaccharide (e.g. lactose, sucrose, maltose), an oligosaccharide or a polysaccharide.

Preferably, R1 is H or Na⁺. Preferably, R2 is the point of attachment to a linker or a polymer. Preferably, R3 is H or OH. Preferably, R4 is H or OH. Preferably, R5 is H or OH. Preferably, R6 is H, OH or O-acetyl. Preferably, R7 is H or OH or acetyl.

In some embodiments of the invention, the first specific binding partner and/or the second specific binding partner comprise a sialic acid linked to a saccharide. For example, the saccharide may be a monosaccharide (e.g. galactose, glucose), a disaccharide (e.g. lactose, sucrose, maltose), an oligosaccharide or a polysaccharide. The sialic acid may, for example, be linked to the saccharide via the C2 carbon of sialic acid (e.g. α2,3- or 2,6-linkage).

Preferably, the linkage is an α2,3- or α2,6-linkage (e.g. α2,3-sialic acid, α2,6-sialic acid, α2,3-sialyllactose or α2,6-sialyllactose).

A sialic acid will, however, always be the terminal group of the first specific binding partner and/or the second specific binding partner.

In some preferred embodiments, the saccharide is lactose, e.g. the first specific binding partner and/or the second specific binding partner is 2,3-sialyllactose or 2,6-sialyllactose, preferably wherein the C2 sialic acid carbon is linked to the C3 or C6 carbons of the galactose moiety of the lactose; and/or preferably wherein the C1 glucose moiety of the lactose is linked to a linker or a polymer, if present.

In some particularly preferred embodiments, the first specific binding partner and/or the second specific binding partner is N-acetyl neuraminic acid (NeuNAc), neuraminic acid, α2,3-sialyllactose or α2,6-sialyllactose.

In some preferred embodiments, the first specific binding partner is (i.e. consists of) a monosaccharide.

In all aspects of the invention, the sialic acid must be exposed in such a manner which allows it to bind to the analyte (e.g. to a coronavirus spike protein), i.e. the sialic acid is not an internal group (e.g. it is not within a polysaccharide).

Preferably, the first specific binding partner and/or second specific binding partner comprises a sialic acid wherein the sialic acid is a terminal group, e.g. at one end of a chain in a disaccharide, oligosaccharide or polysaccharide or other chemical entity.

In particular, the first specific binding partner and/or second specific binding partner is preferably not a glycosylated protein.

The first specific binding partner is linked to a detectable label. This linkage may, for example, be via a linker and/or a polymer. For example, the first specific binding partner/detectable label may have the structure:
first specific binding partner-detectable label,
first specific binding partner-linker-detectable label,
first specific binding partner-polymer-detectable label, or
first specific binding partner-linker-polymer-detectable label.

The second specific binding partner may also be linked to a linker and/or polymer, as defined herein, e.g. in order to facilitate immobilisation of the second specific binding partner. The linker, the polymer or the linker-polymer may be bifunctional.

The function of the linker and/or polymer is to link the first specific binding partner to the detectable label. In some embodiments (e.g. wherein the detectable label is a particle), the linker and/or polymer may be anchored to the detectable label. Any suitable method may be used link the first specific binding partner to the detectable label as long as the linked moieties retain functional activity.

The linker and/or polymer may include carbon atoms and/or heteroatoms (e.g. N, O, S), including linear and/or cyclic moieties, may be branched or unbranched, and may be substituted or unsubstituted. In some embodiments, the backbone (i.e. excluding side chains) of the linker plus polymer (when present) consists of a chain of 40-150 atoms, e.g. 40-80, 80-120 or 120-150 atoms, more preferably about 100 atoms, selected from carbon, nitrogen, sulphur and oxygen.

In some embodiments, the linker and/or polymer is not or does not comprise a saccharide. In some embodiments, the linker and/or polymer is not or does not comprise a polypeptide or a protein. In some embodiments, the linker and/or polymer is not or does not comprise a polynucleic acid. In some embodiments, the linker and/or polymer is not or does not comprise a natural polymer. In particular, in some embodiments, the linker and/or polymer does not comprise over 50, 100 or 1000 sialic acid residues; preferably, the linker and/or polymer is not or does not comprise a sialic acid. The function of the linker is to link the first specific binding partner to the polymer (or to the detectable label).

Common molecular linkers known in the art include amide, ester, thioether, ether, triazole, dihydropyridazine, maleimido, succinimide and hydrazine groups; and streptavidin, neutravidin, biotin, or similar compounds. Non-limiting examples of linkers and linking methods are shown in U.S. Pat. Nos. 9,408,928; 9,993,553; and 10,010,618. Preferably, the linker does not consist or substantially consist of a repeated structure or polymeric structure.

The linker or polymer is covalently attached to the first specific binding partner, preferably at a position as discussed above.

The linker will preferably comprise a terminal functional group which is suitable for linking with the first specific binding partner. The linker may be a bifunctional group.

Preferably, the linker is an amide, triazole, thio-ether or ether bond.

In some preferred embodiments, the first specific binding partner (or second specific binding partner) is linked to a linker, wherein the first specific binding partner-linker (or second specific binding partner-linker) has a structure selected from the group consisting of the following structures:

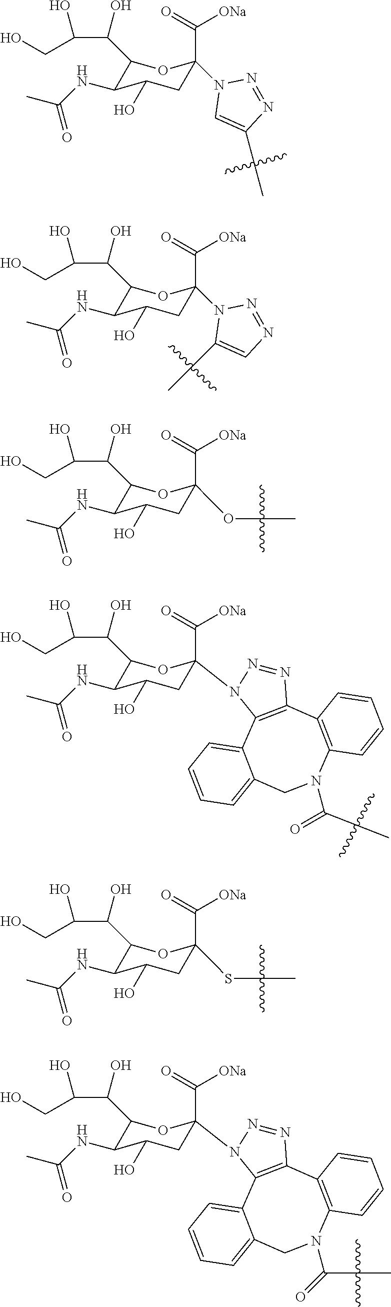

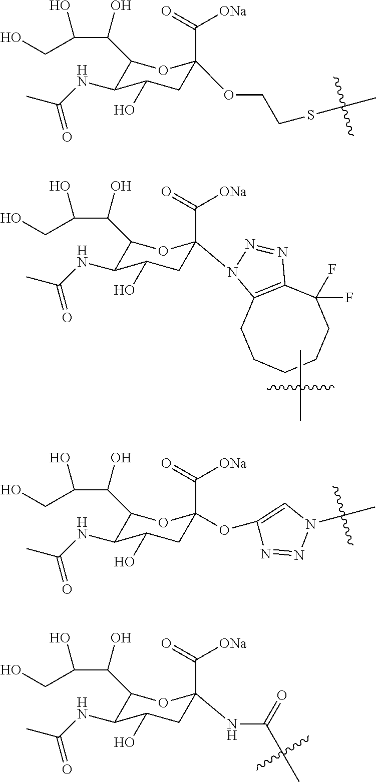

In other preferred embodiments, the first specific binding partner (or second specific binding partner) is linked to a linker, wherein the first specific binding partner-linker (or second specific binding partner-linker) has a structure selected from the group consisting of the following structures:

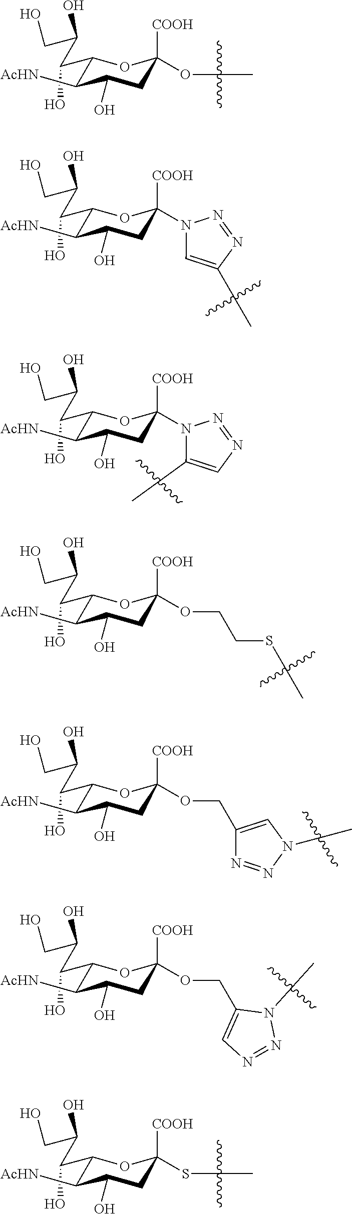

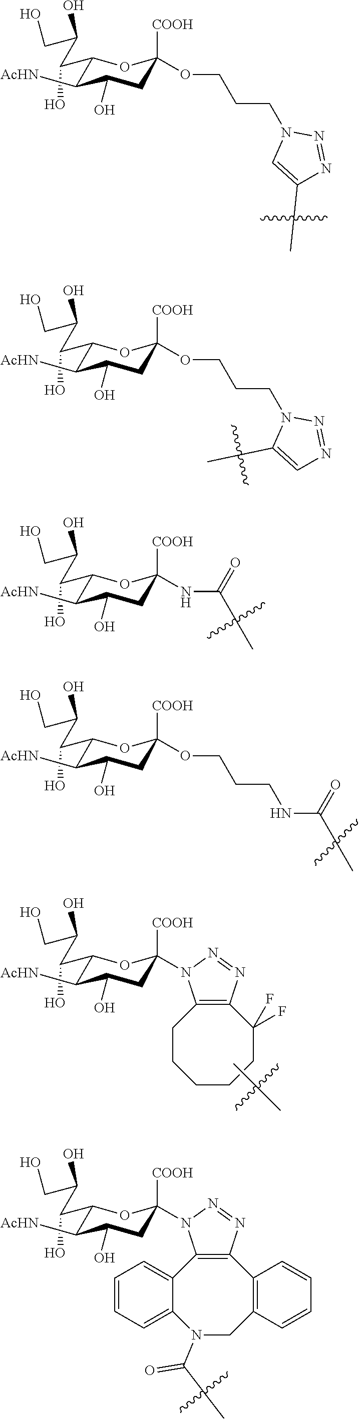

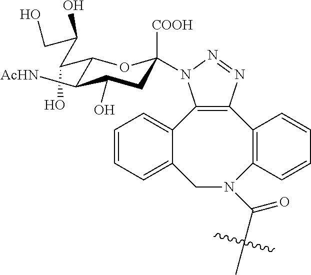

When a linker is present, the polymer links the linker with the detectable label. When a linker is not present, the polymer links the first specific binding partner with the detectable label.

The polymer comprises a polymer or a generally-polymeric material. Preferably, the polymer is a synthetic polymer. In some embodiments, the linker is a water soluble, non-ionic polymer, e.g. polyethylene glycol.

The mechanism of attachment of the polymer to the detectable label will depend on the nature of the polymer and detectable label. Methods of attachment are well known in the art (as discussed further below).

Preferably the polymer comprises [CH₂CH₂O]ₙ or [OCH₂CH₂]ₙ or [N-hydroxyethyl acrylamide]ₙ, wherein n=2-100.

Preferably, the polymer has a structure selected from the group consisting of the following structures:

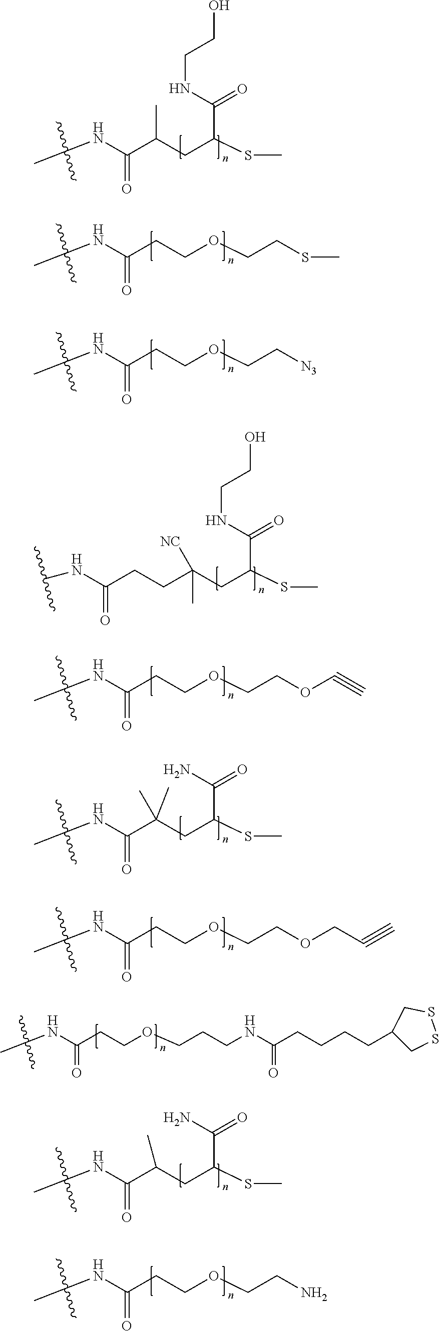

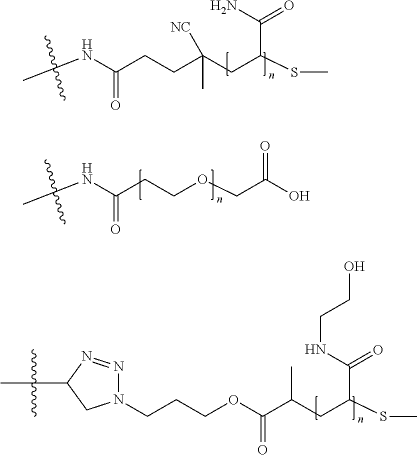

wherein n=1-200. Preferably, n is 5-100, more preferably 30-70, and more preferably 40-60.

In the above structures, the first specific binding partner will be bound to the left-hand end of the structure, and the detectable label will be bound/anchored at the right-hand end of the structure.

In some particularly preferred embodiments, the polymer has the structure:

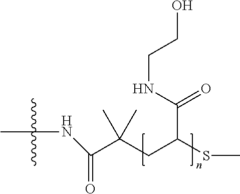

wherein n is 1-200. The detectable label is linked to the —S— group.

Preferably n is 5-100, more preferably 30-70, and more preferably 40-60. In some embodiments, n is 40, 50 or 58.

In some embodiments of this aspect of the invention, the polymer has a number average molecular weight of 4600-7000 g/mol.

In this aspect of the invention, the polymer is attached or anchored to the detectable label via the —S— group.

The first specific binding partner is linked to a detectable label. The label facilitates the detection of the analyte if the first specific binding partner/analyte complex is bound in the detection zone. The label may, for example, be selected from the group consisting of fluorescence tags, dye labels, enzyme reporters, biotin, epitope tags, metal nanoparticles, carbon, coloured latex nanoparticles, magnetic beads, fluorescence beads, and coloured polystyrene beads. Preferably, the label is an optically-detectable marker (i.e. detectable by eye).

In some embodiments, the label has a known density value; this may facilitate the quantification of the marker in the detection zone.

The detectable label may be a multivalent scaffold. As used herein, the term “multivalent scaffold” refers to a support to which a plurality of linkers, as disclosed herein, may be chemically attached or anchored. Examples of multivalent scaffolds include nanoparticles, hyperbranched polymers and cyclodextrins.

In a particularly preferred embodiment, a plurality of polymers are linked to each detectable label (e.g. nanoparticle). Such a plurality of polymers may be used to enhance the affinity of the binding partner-(linker)-polymer-detectable label for the analyte. This plurality can be measured using analytical ultracentrifugation, thermogravimetric analysis or related methods. The presence of polymers can also be confirmed by x-ray photo-electrospectroscopy or NMR spectroscopy. Steric stabilization due to coating of the detectable labels (e.g. nanoparticles) with multiple polymers can also be used to demonstrate successful functionalization with a plurality of polymers, as indicated by resistance to irreversible aggregation in saline or in pharmaceutically-acceptable solutions.

Preferably, the mean number of first specific binding partners attached (via a polymer or linker-polymer) to each detectable label is 2-3000, for example 2-10, 10-25, 25-50, 50-100, 100-150, 150-500, 500-1000, 1000-2000, 2000-3000 or 3000-5000, more preferably 500-3000.

Preferably, the detectable label is a nanoparticle. As used herein, the term “nanoparticle” refers to a nanoscale particle with a size that is measured in nanometres, for example, a nanoscopic particle that has at least one dimension of less than about 200 nm.

Examples of nanoparticles include, by way of example and without limitation, paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes and quantum dots. Other examples of nanoparticles include silicon, carbon and iron oxide nanoparticles.

In particular examples, a nanoparticle is a metal nanoparticle (for example, a nanoparticle of gold, palladium, platinum, silver, copper, nickel, cobalt, iridium, or an alloy of two or more thereof). Nanoparticles can include a core or a core and a shell, as in core-shell nanoparticles.

The size of the nanoparticles may be in a range of from 1 nm to 200 nm, e.g. 5-200 nm, 5-100 nm, 10-20 nm, 20-30 nm, 30-40 nm, 40-50 nm, 50-60 nm, 60-70 nm, 70-80 nm, 80-90 nm or 90-100 nm. In some preferred embodiments, the nanoparticles are 10-40 nm, e.g. about 16 or about 35 nm.

Preferably, the detectable label is a gold nanoparticle (AuNP). In LFDs which utilise gold nanoparticles as the detectable label, the binding of the analyte in the detection zone results in the appearance of a red mark.

Preferably, the average size of the gold nanoparticles is 5-50 nm in diameter, more preferably 12-40 nm, and most preferably about 16 nm or about 35 nm in diameter.

Methods of producing gold nanoparticles are well known in the art (e.g. Zhao et al. Coordination Chemistry Reviews, vol. 257, issues 3-4, February 2013, pages 638-665).

Any suitable method may be used to link the linker to the gold nanoparticle as long as the linked moieties retain functional activity. Non-limiting examples of linkers and linking methods are shown in U.S. Pat. Nos. 9,408,928; 9,993,553; and 10,010,618. Common molecular linkers known in the art include a maleimide or succinimide group, streptavidin, neutravidin, biotin, or similar compounds. For example, functional groups may be used to covalently-link or electrostatically-link the linker to the nanoparticles. Such functional groups include any group that can be reacted with another compound to form a covalent linkage between the linker and the nanoparticle. Examples of such functional groups include, but are not limited to, carboxylic acids and carboxylic acid salt derivatives, acid halides, sulfonic acids and sulfonic acid salts, anhydride derivatives, hydroxyl derivatives, amine and amide derivatives, silane derivations, phosphate derivatives, nitro derivatives, succinimide and sulfo-containing succinimide derivatives, halide derivatives, alkene derivatives, morpholine derivatives, cyano derivatives, epoxide derivatives, ester derivatives, carbazole derivatives, azide derivatives, alkyne derivatives, acid containing sugar derivatives, glycerol analogue derivatives, maleimide derivatives, protected acids and alcohols, acid halide derivatives, and combinations thereof. The functional groups can be substituted or unsubstituted.

In some particularly-preferred embodiments, the
first specific binding partner-linker-polymer-detectable label
   has one of the following structures:

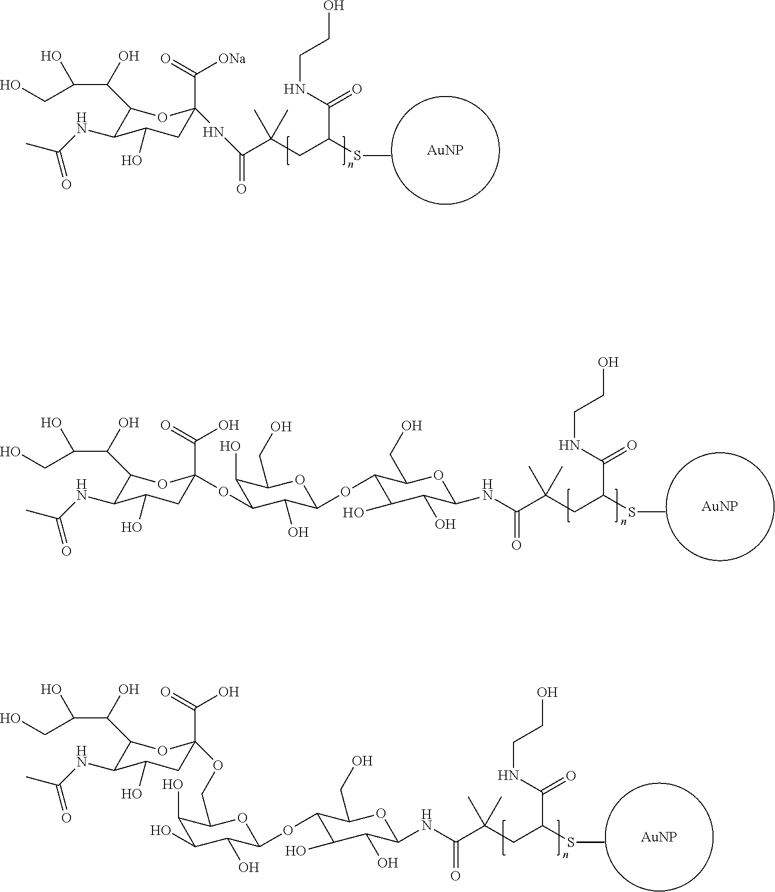

wherein n=30-70, preferably 40-60, more preferably about 48, 50 or 58. AuNP represents a gold nanoparticle.

In embodiments wherein the detectable label is a nanoparticle, the nanoparticles are preferably colloidally-stable. A colloid is a mixture in which microscopically-dispersed insoluble or soluble particles are suspended throughout another substance. In the context of the current invention, the detectable label may be insoluble; and the compounds of the invention will, in use, be dispersed within an aqueous solution.

Some embodiments of the polymers disclosed herein provide enhanced colloidal stability to the first specific binding partner-(linker)-polymer-detectable label compounds.

Colloidally-stable means the nanoparticle compounds are not significantly aggregated (i.e. more than 50%, 60%, 70%, 80% or 90% aggregated) upon storage at temperatures between 4 and 50° C. (e.g. at 21° C.) or can be re-dispersed through physical agitation.

Colloidal stability may be determined in a pharmaceutically-relevant media. Such media include buffers such as phosphate buffered saline (PBS) and HEPES, either with or without a detergent (such as SDS) or blocking agents (PVP, PEG, BSA, casein, polysaccharides).

Colloidal stability can be judged by those skilled in the art using method such as dynamic light-scattering and turbidimetry. Furthermore, gold nanoparticle aggregation can be monitored by UV-visible spectroscopy by a shift in the surface plasmon resonance maxima.

A second specific binding partner is immobilised in the detection zone. Preferably, the second specific binding partner consists of or comprises a sialic acid, as defined herein. In embodiments of the invention wherein the first and second binding partners are both a sialic acid, the sialic acids may be the same or different.

In some embodiments of the invention, the first or second specific binding partner may comprise a ligand (e.g. other than a sialic acid) which binds to the analyte.

The ligand may be a ligand which binds specifically to the analyte or non-specifically to the analyte. For example, in embodiments wherein the analyte is a virus, the ligand may be a reagent which binds non-specifically to viruses (e.g. a virus-binding lectin, such as a C-type lectin receptor, preferably DC-Sign; or Staphylococcus A protein).

Preferably, the analyte is a virus or virus protein, preferably a coronavirus or a coronavirus protein (e.g. spike protein). In such embodiments, the ligand is a ligand which binds to coronaviruses or a coronavirus proteins, either specifically or non-specifically.

Preferably, the ligand is an antibody which binds specifically or non-specifically to the analyte.

The antibody may, for example be a whole antibody, a monoclonal antibody, an antibody fragment, a humanized antibody, a single chain antibody, a defucosylated antibody, an antibody mimetic or a bispecific antibody. Antibody fragments include a UniBody, a domain antibody and a Nanobody. Antibody mimetics include an Affibody, a DARPin, an Anticalin, an Avimer, a Versabody and a Duocalin.

Preferably, the antibody is a monoclonal antibody.

In some preferred embodiments, the ligand is an anti-coronavirus antibody, more preferably an anti-SARS-CoV-2 antibody which binds specifically to SARS-CoV viruses or SARS-CoV-2 proteins. Such antibodies are available from Sino Biological (UK) and Abcam.

The method of immobilisation of the ligand in the detection zone will depend on the nature of the ligand and the detection zone substrate. Such methods are well known in the art (e.g. Bahadir, E. B.; Sezgintürk, M. K. Lateral Flow Assays: Principles, Designs and Labels. TrAC Trends Anal. Chem. 2016, 82, 286-306; and Brown, M. C. Antibodies: Key to a Robust Lateral Flow Immunoassay. In Lateral Flow Immunoassay; 2009; pp. 59-74).

Method of linking ligands (e.g. antibodies) to particles (e.g. gold particles) are well known in the art, e.g. the use of biotin-labelled antibodies which are bound to avidin/streptavidin-coated particles (see also Yi-Cheun Yeh et al., “Gold Nanoparticles: Preparation, Properties, and Applications in Bionanotechnology”, Nanoscale. 2012 Mar. 21; 4(6): 1871-1880).

In some embodiments, the sensitivity of the method or device of the invention may be improved by silver staining any virus (e.g. coronavirus) particles or proteins, e.g. any virus (e.g. coronavirus) particles or proteins which are bound in the detection zone, or control zone or test line.

In yet another embodiment, the invention provides a kit comprising:
(A) an aqueous composition comprising a first specific binding partner linked to a detectable label, and
(B) a substrate upon which a second specific binding partner is immobilised; characterised in that
(i) the first specific binding partner comprises a sialic acid and the second specific binding partner is an anti-SARS-CoV-2 antibody; or
(ii) the first specific binding partner is an anti-SARS-CoV-2 antibody and the second specific binding partner comprises a sialic acid.

Preferably, the substrate is a LFD as disclosed herein.

The aqueous composition may, for example, be in the form of a conical tube (e.g. Eppendorf tube or PCR tube) or a multi-well plate. Examples of aqueous compositions include phosphate-buffered saline or HEPES, optionally additionally including one or more of a pharmaceutically-acceptable salt, a blocking agent (e.g. BSA, poly(vinylpyrrolidone), PEG) and a detergent (e.g. SDS). Other polymers may also be included in the aqueous composition (e.g. poly(hydroxyl ethyl acrylamide), poly(ethylene glycol), casein) to modulate the density of the linkers and/or polymers to optimise the binding and functional outputs of the assay.

Examples of detectable labels include those disclosed herein. More preferably, the detectable label is a detectable label as disclosed herein, most preferably a gold nanoparticle, optionally linked to a polymer as disclosed herein.

Preferably, the aqueous solution has an optical density (absorbance at 520 nm) between 0.1 and 10, more preferably between 0.1 and 5, and most preferably OD=about 1.

Examples of anti-SARS-CoV-2 antibodies include those disclosed herein.

In yet a further embodiment, the invention provides a method of determining the presence of SARS-CoV-2 particles or SARS-CoV-2 proteins in a test sample, the method comprising the steps:
(a) contacting a substrate upon which a second specific binding partner (as defined herein) is immobilised with a test sample with an aqueous composition comprising a first specific binding partner linked to a detectable label (as defined herein); and
(b) detecting the presence or absence of bound label on the substrate;
   wherein the presence of bound label on the substrate is indicative of the presence of SARS-CoV-2 particles or SARS-CoV-2 proteins in the test sample.

The substrate and the test sample may be contacted with the composition in either order.

As a minimum, at least the zone of the substrate to which the first specific binding partner is immobilised will be required to be contacted with the composition (and the test sample).

The polymers of the invention are particularly colloidally-stable. In yet a further embodiment, therefore, the invention provides a compound having the structure:
sugar-(linker)-polymer-detectable label,
   wherein the sugar is preferably a monosaccharide, disaccharide or trisaccharide, the linker is as defined herein and is optionally present, and the polymer is a structure selected from the following structures:

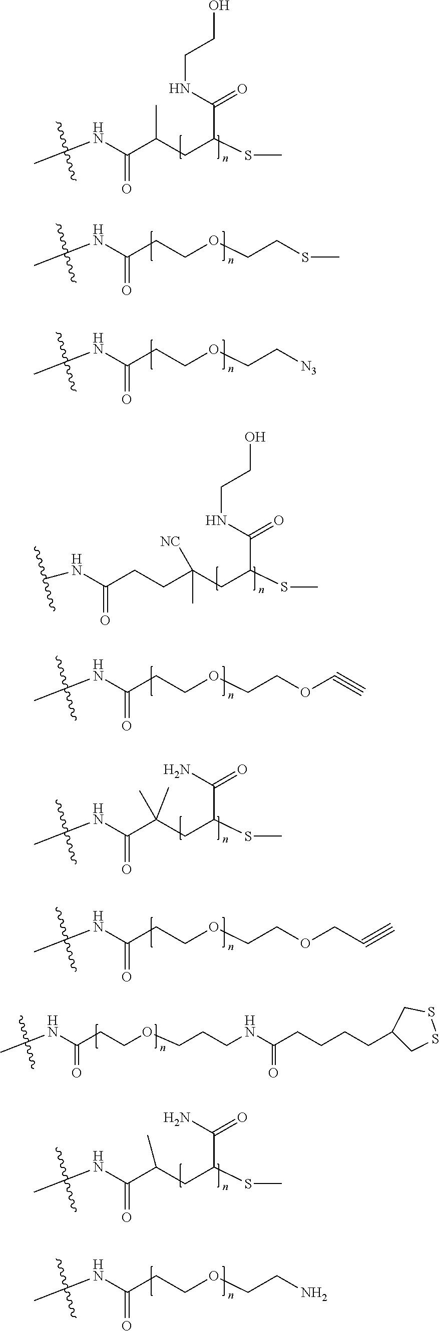

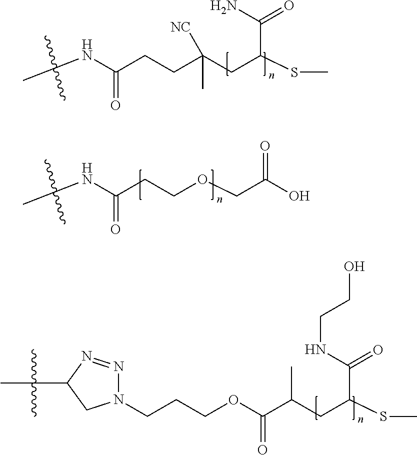

preferably,

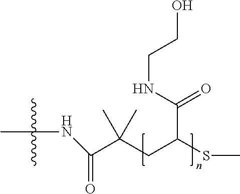

wherein n=1-200, preferably, 5-100, more preferably 30-70, and most preferably 40-60; and the detectable label is as defined herein, preferably a gold nanoparticle.

In another embodiment, the invention provides a flow device (preferably a lateral flow device or a flow-though device) for detecting the presence of an analyte in a test sample, the device comprising a conjugate zone and a detection zone, wherein:
(a) the conjugate zone comprises a first specific binding partner for the analyte, wherein the first specific binding partner is linked to a detectable label, and wherein the first specific binding partner is not immobilised in the conjugate zone; and
(b) the detection zone comprises a second specific binding partner for the analyte, wherein the second specific binding partner is immobilised in the detection zone,
   characterised in that the first specific binding partner/detectable label has the structure: sugar-(linker)-polymer-detectable label, as defined above.

Preferably, the analyte is a virus particle or a virus surface protein, e.g. a sugar-binding virus or a respiratory virus, more preferably a coronavirus particle or a coronavirus spike protein, and most preferably a SARS-CoV-2 virus particle or a SARS-CoV-2 S1 protein.

The methods of the invention may also be used, mutatis mutandis, to detect non-coronavirus viruses, wherein the sialic acid moiety and/or the linker-sialic acid moiety is tailored for the specific detection of the (non-coronavirus) virus.

The table below shows selected, non-exhaustive, examples of viruses which can bind sialic acid terminated glycans.

| Virus | Sialic Acid Binder |
|---|---|
| Human Coronavirus OC43 [22, 23] | 9-O-Acetylated sialic acids |
| Human Coronavirus HKU1 [22] | 9-O-Acetylated sialic acids |
| Middle East Respiratory Syndrome | α2-3- & α2-6-linked sialyl |
| coronavirus (MERS-CoV) [24] | sequences |
| Adenovirus 37 [25, 26] | α2-3-linked sialyl lactose |
| H1N1 virus, A/Hamburg/5/2009 | α2-3- & α2-6-linked sialyl |
| (Ham/09) [27] | sequences |
| H1N1 virus, A/Memphis/14/96-M | α2-6-linked sialyl sequences |
| (Mem/96) [27] | |
| H3N2, A/Wyoming/03/03 X-147 | Neu5Acα2-6Galα1-4GlcNAc |
| [28] | terminated sequences |
| Mumps virus (MuV) [29] | α2-3-sialyl sequences |
| hPIV-1 & hPIV-3 [30] | Neu5Acα2-3Galα1-4GlcNAc- |
| | terminated ganglioside |
| Norovirus GII.3 (Chron1) [31] | Sialyl Lewis X |

In yet further embodiments, therefore, the invention provides methods and devices comprising the features as disclosed herein, wherein the methods and devices are for determining the presence of virus particles or virus proteins (instead of coronavirus particles and coronavirus proteins).

In particular, the invention provides a method of determining the presence of virus particles or virus proteins in a test sample, the method comprising the steps:

(a) contacting the test sample with a first specific binding partner, wherein the first specific binding partner comprises a terminal sialic acid, and wherein the first specific binding partner is linked to a detectable label; and
(b) detecting the presence or absence of detectable label which is bound to the test sample,
wherein the presence of detectable label which is bound to the test sample is indicative of the presence of a virus particle or virus protein in the test sample.

Preferably, the viruses are selected from the group consisting of coronaviruses, adenoviruses, influenza viruses, mumps viruses, parainfluenza viruses and noroviruses.

In some embodiments, the virus is an influenza virus, preferably H3, more preferably H3N2.

More preferably, the virus to be detected and the corresponding sialic acid are selected from the above table or from the following table:

| Virus | Sialic Acid Binder |
|---|---|
| Human Coronavirus | 9-O-Acetylated sialic acids |
| Middle East Respiratory | α2-3- & α2-6-linked sialyl sequences |
| Syndrome coronavirus | |
| (MERS-CoV) | |
| Adenovirus | α2-3-linked sialyl lactose |
| H1N1 virus | α2-3- & α2-6-linked sialyl sequences |
| H3N2 virus | Neu5Acα2-6Galβ1-4GlcNAc terminated |
| | sequences |
| Mumps virus (MuV) | α2-3-sialyl sequences |
| hPIV-1 or hPIV-3 | Neu5Acα2-3Galα1-4GlcNAc-terminated |
| | ganglioside |
| Norovirus | Sialyl Lewis X |

The disclosure of each reference set forth herein is specifically incorporated herein by reference in its entirety.

### BRIEF DESCRIPTION OF THE FIGURES

FIG. 1. A) Sequence alignment of hypothesized sialic acid binding sites of coronaviruses (SEQ ID NOs: 1-9); B) Model showing the sialic acid binding site for SARS-CoV-2 spike protein assembly and the 51, S2 domains; C) MERS sialic acid binding site in complex with 2,3-sialyllactose.

FIG. 2. Synthesis of polymer-stabilized glycosylated nanoparticles.

FIG. 3. ¹H NMR spectrum of DP40 PHEA.

FIG. 4. ¹H NMR spectrum of DP50 PHEA.

FIG. 5. ¹H NMR spectrum of DP58 PHEA.

FIG. 6. ¹⁹F NMR spectra of PFP-PHEA40 before (lower) and after (upper) reaction with 2-amino NeuNAc.

FIG. 7. TEM images (left) and histograms (right) of citrate stabilized AuNPs. A) 16 nm AuNP and B) 35 nm AuNP. Histograms from analysis of analysis of >100 particles.

FIG. 8. Increased stability to saline concentration due to polymer coating. Top row are UV-visible traces upon addition of indicated saline gradient. Bottom row is dynamic light scattering in saline.

FIG. 9. Biolayer interferometry analysis of SARS-CoV-2 spike protein with glyconanoparticles. A) Screening using PHEA₅₀@AuNP₃₅ at OD=1; Dose dependent binding of NeuNAc-PHEA₄₀ using B) @AuNP16 and C) @AuNP35. D) Binding curves summary.

FIG. 10. Lateral flow analyses of NeuNAcPHEAx@AuNPy particles. A) ‘Half’ lateral flow assays setup. Effect of polymer chain length and particle size on lateral flow binding (B) and signal:noise analysis (C). D) Selectivity of NeuNAcPHEA₄₀@AuNP₃₅ against a panel of lectins (inset example LFD strips). E) Selectivity of NeuNAcPHEA₄₀@AuNP₃₅ against S1 protein from different coronavirus strains.

FIG. 11. Specificity and limit of detection of SARS-CoV-2, S1 protein versus NeuNAc and galactose-functional nanoparticles. A) flow strips and B) signal intensity from image analysis.

FIG. 12. Schematic showing set-up of a flow assay where specimens are applied as the test line.

FIG. 13. Examples of complete flow devices using positive or negative COVID-19 patient specimens, where the specimen was deposited as the test line.

FIG. 14. Device performance using heat-inactivated primary patient swabs after silver staining step (positive result is test and control line being visible). Confusion matrices after silver staining. Sensitivity=TP/(TP+FN); Specificity=TN/(TN+FP); PPV=TP/(TP+FP); NPV=TN/(TN+FN). TP=true positive; TN=true negative; FN=false negative; FP=false positive.

FIG. 15. Examples of complete flow devices where the indicated hemagglutinin has been deposited as a test line.

FIG. 16. Devices showing the detection of the Denmark, UK and South African variants of SARS-COV-2.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Sialic Acid Binding Site

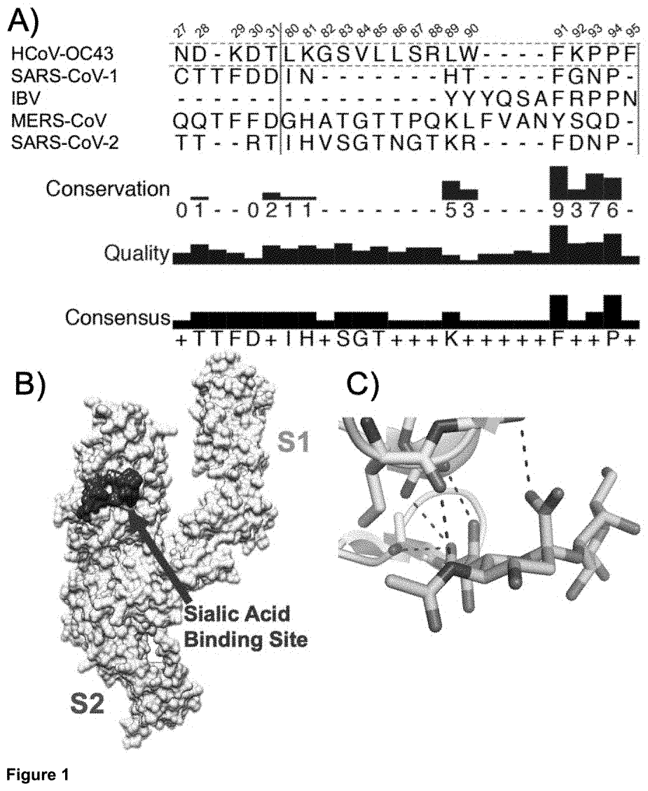

FIG. 1A shows the multiple sequence alignment of spike proteins from SARS-CoV-1, IBV, MERS-CoV, and SARS-CoV-2 with respect to the known sialic acid binding grove sequence of HCoV-0C43.²⁹ There are no clear conserved residues between the sequences, but Phe91 and Pro94 are common to all sequences apart from the MERS sequence. This is in marked contrast to the spike S protein in general, which is highly conserved.³⁰ This lack of sequence homology within the sialic acid binding grove may contribute to the virus' ability to cross between species.³¹

FIG. 1B shows a model illustrating the sialic acid binding site for SARS-CoV-2 spike protein assembly and the S1 and S2 domains.

FIG. 1C shows the MERS sialic acid binding site in a complex with 2,3-sialyllactose, showing that only the sialic acid unit, not the lactose unit, engages with the binding site.

This opens up the possibility that a sialic acid may be a reasonable target for coronavirus detection by LFD.

Sequence Alignment Information for FIG. 1

The multiple sequence alignment was done using Clustal Omega1 with the following GenBank accession numbers:

| Coronavirus | GenBank accession numbers |
|---|---|
| HCoV-OC43 | AAT84354.1 |
| SARS-CoV-1 | AAP13441.1 |
| IBV-CoV | AAW33786.1 |
| MERS-CoV | AYM48030.1 |
| SARS-CoV-2 | QHD43416.1 |

### Example 2: Production of Linkers and Nanoparticles

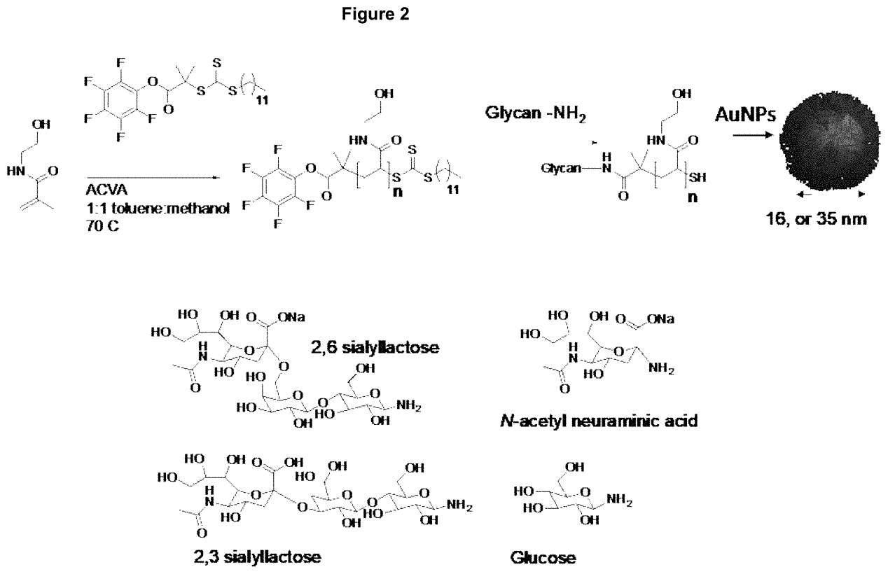

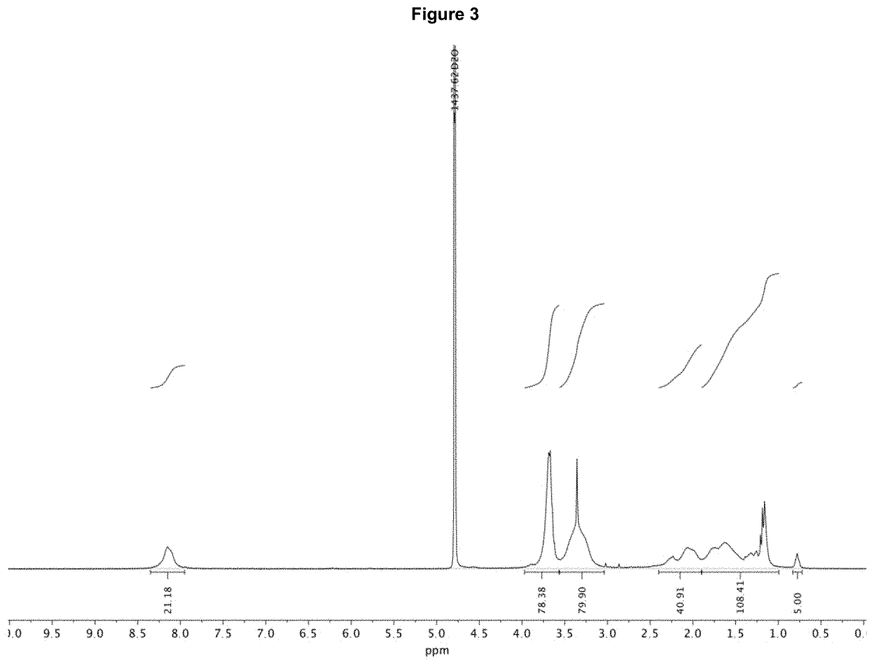

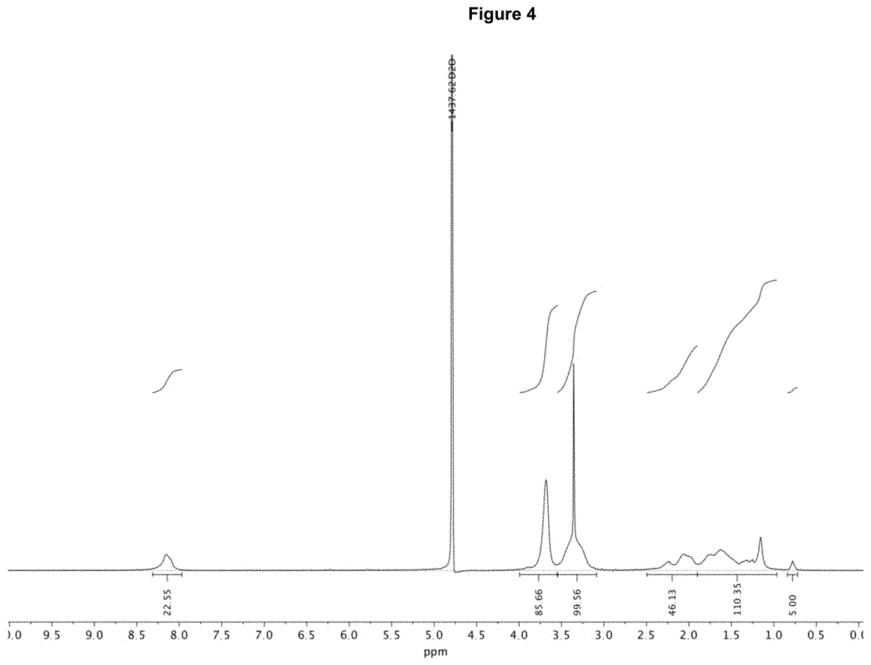

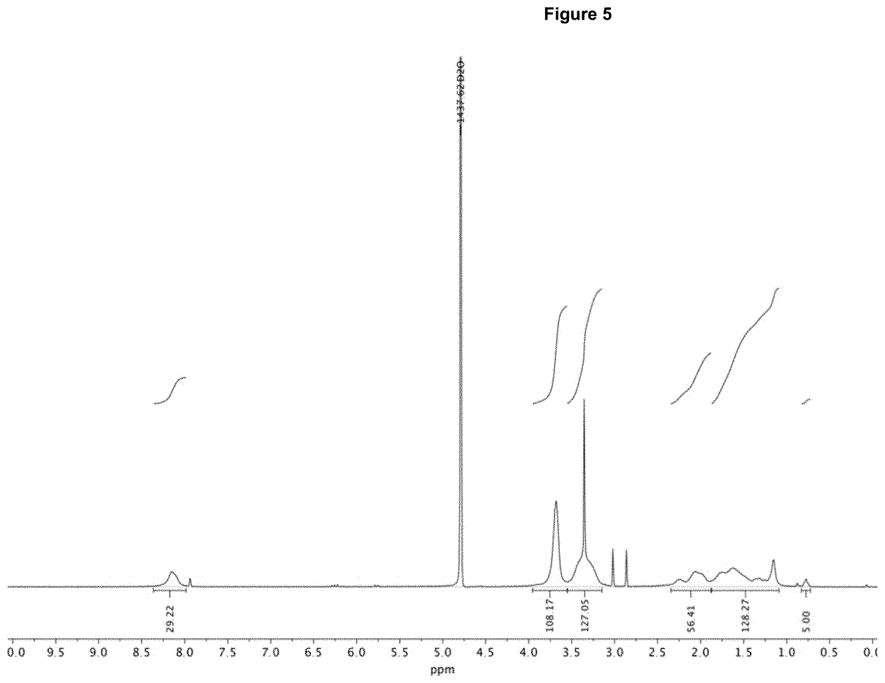

RAFT polymerization was used to obtain poly(N-hydroxylethyl acrylamide), PHEA, which was capable of capturing amino-terminated glycans at the ω-terminal pentafluorophenyl (PFP) group and conjugating to gold particles at the α-terminal thiol, FIG. 2/Table 1.^{19,20} These were characterized by NMR (FIGS. 3-5).

The PHEAs had dispersities below 1.3 as determined by size exclusion chromatography, Table 1. PHEAs lengths were selected based on performance (data not shown) in initial lateral flow screening assays.

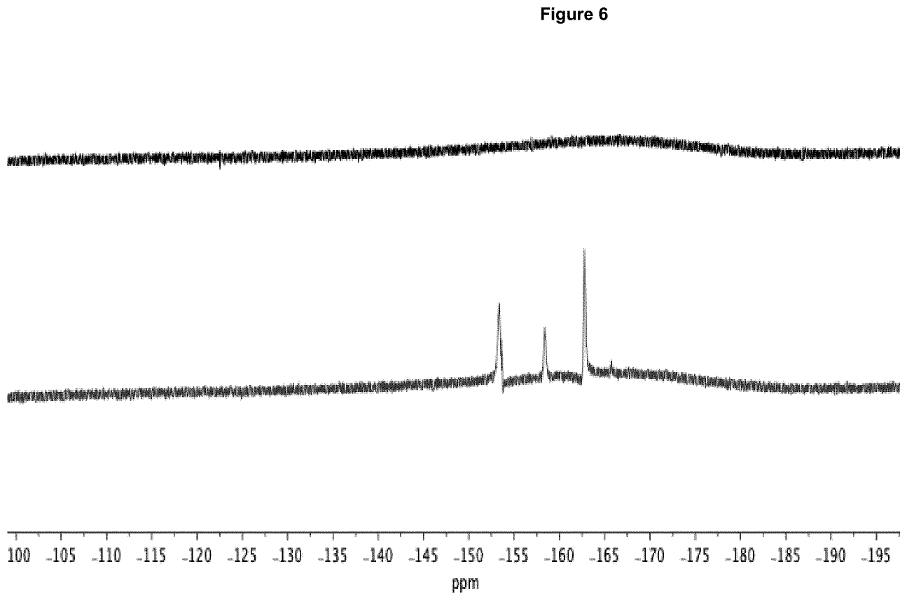

Amino-glycans were synthesized by reduction of anomeric azides and their conjugation to polymers by displacement of the PFP group was confirmed by ¹⁹F NMR (FIG. 6).

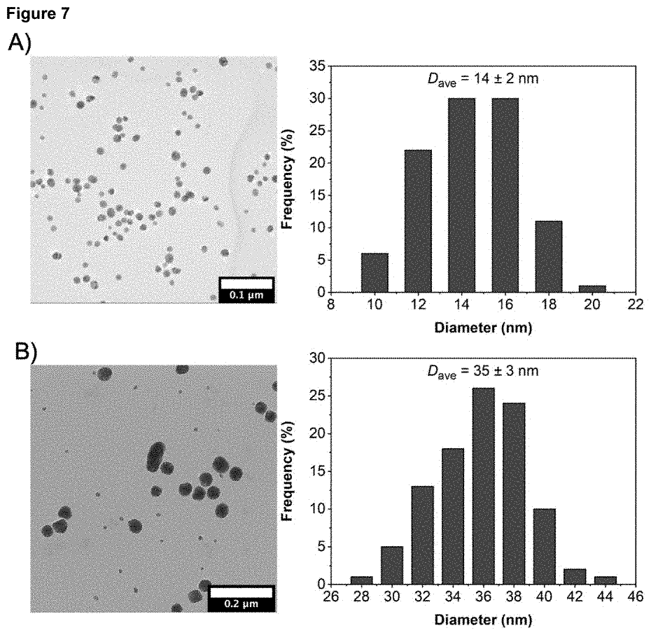

Polymers were assembled onto citrate-stabilized gold nanoparticles and excess ligand removed by centrifugation/resuspension and were characterized by UV-Vis, dynamic light scattering (DLS) and transmission electron microscopy (TEM) (FIG. 7) shown in Table 2. XPS (X-ray photoelectron spectroscopy confirming surface coating).

TABLE 1
Polymers Synthesized

| | M:CTA | Mn₍ₜₕₑₒ₎^{(a)} | Mn_{(SEC)}^{(b)} | Mn_{(NMR)}^{(c)} | Ð^{(b)} |
|---|---|---|---|---|---|
| Code | (-) | (g · mol⁻¹) | (g · mol⁻¹) | (g · mol⁻¹) | (-) |
| PHEA40 | 20 | 2800 | 5100 | 5000 | 1.19 |
| PHEA50 | 25 | 3400 | 6400 | 5500 | 1.27 |
| PHEA58 | 30 | 4000 | 7200 | 6700 | 1.26 |

^{(a)}Estimated from [M]:[CTA];
^{(b)}From DMF SEC versus PMMA standards;
^{(c)1}H NMR end-group analysis.

TABLE 2
Nanoparticle Characterization

| | | A_{SPR}/ | | | |
|---|---|---|---|---|---|
| | UVmax^{(a)} | A₄₅₀^{(b)} | Dh^{(c)} | D_{h (DLS)}^{(d)} | D_{(TEM) (e)} |
| Code | (nm) | (-) | (nm) | (nm) | (nm) |
| AuNP₁₆ | 519 | 1.64 | 16 | 20.7 ± 0.8 | 14 ± 2 |
| NeuNAc-PHEA₅₀AuNP₁₆ | 527 | 1.66 | 16 | 40.9 ± 0.5 | |
| NeuNAc-PHEA₅₈AuNP₁₆ | 526 | 1.68 | 18 | 44.2 ± 0.8 | |
| AuNP₃₅ | 526 | 1.91 | 35 | 34.5 ± 0.5 | 35 ± 3 |
| NeuNAc-PHEA₅₀AuNP₃₅ | 531 | 1.98 | 45 | 46.2 ± 0.7 | |
| NeuNAc-PHEA₅₈AuNP₃₅ | 531 | 1.99 | 45 | 55.3 ± 0.8 | |

^{(a)}SPR absorption maximum;
^{(b)}Absorbance ratio of SPR to 450 nm;
^{(c)}Estimated from UV-Vis²¹;
^{(d)}From dynamic light scattering;
₍ₑ₎ From TEM, from average of >100 particles, showing ±S.D.
NeuNAc = N-acetyl neuraminic acid.
AuNP = gold nanoparticle; diameters shown in subscript in nm.

NMR Spectroscopy

¹H-NMR, ¹³C-NMR and ¹⁹F-NMR spectra were recorded at 300 MHz or 400 MHz on a Bruker DPX-300 or DPX-400 spectrometer respectively, with chloroform-d (CDCl₃) or deuterium oxide (D₂O) as the solvent. Chemical shifts of protons are reported as δ in parts per million (ppm) and are relative to either CDCl₃ (7.260) or D₂O (4.790).

Mass Spectrometry

Low resolution mass spectra (LRMS) were recorded on a Bruker Esquire 2000 spectrometer using electrospray ionisation (ESI). M/z values are reported in Daltons.

FT-IR Spectroscopy

Fourier Transform-Infrared (FT-IR) spectroscopy measurements were carried out using an Agilent Cary 630 FT-IR spectrometer, in the range of 650 to 4000 cm⁻¹.

Size Exclusion Chromatography

Size exclusion chromatography (SEC) analysis was performed on an Agilent Infinity II MDS instrument equipped with differential refractive index (DRI), viscometry (VS), dual angle light scatter (LS) and variable wavelength UV detectors. The system was equipped with 2×PL gel Mixed D columns (300×7.5 mm) and a PLgel 5 μm guard column. The mobile phase used was DMF (HPLC grade) containing 5 mM NH₄BF₄ at 50° C. at flow rate of 1.0 mL·min⁻¹. Poly(methyl methacrylate) (PMMA) standards (Agilent EasyVials) were used for calibration between 955,000-550 g·mol⁻¹. Analyte samples were filtered through a nylon membrane with 0.22 μm pore size before injection. Number average molecular weights (Mₙ), weight average molecular weights (M_{w}) and dispersities (ÐM=M_{w}/Mₙ) were determined by conventional calibration and universal calibration using Agilent GPC/SEC software.

Dynamic Light Scattering

Hydrodynamic diameters (Dh) and size distributions of particles were determined by dynamic light scattering (DLS) using a Malvern Zetasizer Nano ZS with a 4 mW He—Ne 633 nm laser module operating at 25° C. Measurements were carried out at an angle of 173° (back scattering), and results were analysed using Malvern DTS 7.03 software. All determinations were repeated 5 times with at least 10 measurements recorded for each run. Dₕ values were calculated using the Stokes-Einstein equation where particles are assumed to be spherical.

UV-Vis Spectroscopy

Absorbance measurements were recorded on an Agilent Cary 60 UV-Vis Spectrophotometer and on a BioTek Epoch microplate reader.

Materials

All chemicals were used as supplied unless otherwise stated. N-Hydroxyethyl acrylamide (97%), 4,4′-azobis(4-cyanovaleric acid) (98%), mesitylene (reagent grade), triethylamine (>99%), sodium citrate tribasic dihydrate (>99%), gold(III) chloride trihydrate (99.9%), ammonium carbonate (reagent grade), potassium phosphate tri basic (≥98%, reagent grade), potassium hexafluorophosphate (99.5%), deuterium oxide (D2O, 99.9%), Deuterochloroform (CDCl3, 99.8%), diethyl ether ((≥99.8%, ACS reagent grade), sodium azide (≥99.5%, reagent plus grade), hydrazine hydrate (50-60%), methanol (≥99.8%, ACS reagent grade), Amberlite® 1R120 (H+ form), toluene (≥99.7%), Tween-20 (molecular biology grade), HEPES, PVP40 (poly(vinyl pyrrolidone)400 (Average Mw˜40,000)), sucrose (Bioultra grade), carbon disulphide (≥99.8%), acetone (≥99%), 1-dodecane thiol (≥98%), pentafluorophenol (≥99%, reagent plus) were all purchased from Sigma-Aldrich. 3′sialyllactose and 6′sialyllactose were purchased from Carbosynth. Distilled water used for buffers was MilliQ grade 18.2 mΩ resistance. Soybean agglutinin, Ricinus communis Agglutinin I (RCA120), Sambucus niger Lectin, Ulex europaeus Agglutinin I and wheat germ agglutinin were purchased from Vector Laboratories. 3′-sialyl lactose-BSA (3 atom spacer, NGP0702), 6′sialyl lactose-BSA (3 atom spacer, NGP0706) and N-acetylneuraminic acid-BSA (6 atom spacer, NGP6111) were purchased from Dextra Laboratories. 2-azido-2-deoxy-N-acetyl-D-neuraminic acid was a gift from Iceni Diagnostics Ltd, and reduced to the amine using hydrazine/palladium. SARS Coronavirus Spike Glycoprotein (S1), His-Tag (HEK293)-SARS-CoV-S1 spike protein was purchased from the Native Antigen Company, or provided by Dr Anne Straube, UoW.

Polymer Synthesis Using 2-Hydroxyethyl Acrylamide (Actual Polymer DP40 by SEC)

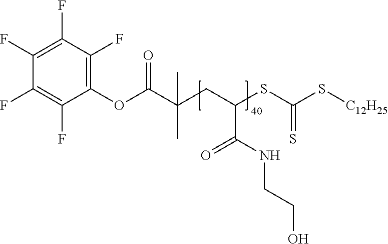

2.0 g (17.37 mmol) of 2-hydroxyethyl acrylamide, 0.043 g (0.15 mmol) of ACVA and 0.368 g (0.69 mmol) of PFP-DMP was added to 16 ml 1:1 toluene:methanol and degassed with nitrogen for 30 minutes. The reaction vessel was stirred and heated to 70° C. for 2 hours. The solvent was removed under vacuum. The crude product was dissolved in the minimum amount of methanol. Diethyl ether cooled in liquid nitrogen was added to the methanol to form a precipitate. The mixture was centrifuged for 2 minutes at 13 krpm and the liquid decanted off. The solid was dissolved in methanol and removed under vacuum to give a yellow crystalline solid.

δ_{H} (300 MHz, D₂O) 8.35-7.95 (21H, m, NH) 3.97-3.56 (78H, m, NHCH₂), 3.56-3.03 (80H, m, CH₂OH & SCH₂), 2.41-1.90 (41H, m, CH₂CHC(O) & C(CH₃)₂), 1.90-0.99 (108H, m, CH₂CHC(O) & CH₂CH₂CH₂CH₂CH₂CH₂CH₂CH₂CH₂CH₂CH₃), 0.83-0.72 (5H, m, CH₂CH₃) δ_{F} (300 MHz, D₂O) −152.0-−164.3 (5F, m, C₆F₅). FTIR (cm⁻¹) —3263.3 (OH, broad), 3088.1 & 2924.1 (C(O)NH and NH), 1638.2 & 1541.3 (C(O)NH) Yield −73%

Representative DP40 Poly(N-hydroxyethyl acrylamide) Glycan Functionalisation Using 2-Amino-2-deoxy-N-acetyl-D-neuraminic Acid

0.2 g (0.039 mmol) of poly(2-hydroxyethyl acrylamide)40 and 0.078 mmol of glycan were added to 20 ml of DMF containing 0.05 M TEA. The reaction was stirred at 50° C. for 16 hours. Solvent was removed under vacuum. The crude product was dissolved in the minimum amount of methanol. Diethyl ether cooled in liquid nitrogen was added to the methanol to form a precipitate. The mixture was centrifuged for 2 minutes at 13 krpm and the liquid decanted off. The solid was dissolved in methanol and solvent removed under vacuum to give an orange/brown crystalline solid. Loss of fluorine signal in the 19F NMR was used to indicate the reaction had gone to completion. δH (300 MHz, D2O) 8.21-7.99 (25H, m, NH), 4.10-3.57 (˜90H, m, NHCH2 & glycan protons), 3.57-2.99 (˜82H, m, CH2OH & SCH2 & glycan protons), 2.40-1.87 (50H, m, CH2CHC(O), C(CH3)2 & & glycan protons), 1.87-0.99 (110H, m, CH2CHC(O) & CH2CH2CH2CH2CH2CH2CH2CH2CH2CH3 & glycan protons), 0.86-0.74 (5H, m, CH2CH3).

Citrate-stabilised 16 nm Gold Nanoparticle Synthesis

To 500 ml of water was added 0.163 g (0.414 mmol) of gold(III) chloride trihydrate, the mixture was heated to reflux and 14.6 ml of water containing 0.429 g (1.46 mmol) of sodium citrate tribasic dihydrate was added. The reaction was allowed to reflux for 30 minutes before cooling to room temperature over 3 hours. The solution was centrifuged at 13 krpm for 30 minutes and the pellet resuspended in 40 ml of water to give an absorbance at 520 nm of ˜1Abs.

Gold Nanoparticle Polymer Coating 16 nm

100 mg of glycopolymer was agitated overnight with 10 ml of 16 nm AuNPs ˜1 Abs at UVmax. The solution was centrifuged at 13 krpm for 30 minutes and the pellet resuspended in 10 ml of water, the solution was centrifuged again at 13 krpm for 30 minutes and the pellet resuspended in 1 ml aliquots and centrifuged at 14.5 krpm for 10 minutes. The pellets were combined into a 1 ml solution with an absorbance at 520 nm of ˜10 Abs.

Gold Nanoparticle Polymer Coating 35 nm

100 mg of glycopolymer was agitated overnight with 10 ml of 35 nm AuNPs ˜1 Abs at UVmax. The solution was centrifuged at 8 krpm for 30 minutes and the pellet resuspended in 10 ml of water, the solution was centrifuged again at 8 krpm for 30 minutes and the pellet resuspended in 1 ml aliquots and centrifuged at 8 krpm for 10 minutes. The pellets were combined into a 1 ml solution with an absorbance at UVmax of ˜10 Abs.

### Example 3: Nanoparticle Multivalency and Colloidal Stability

Table 3 shows XPS characterization of the nanoparticles both as synthesized (with citrate capping ligands) and after functionalization with polymers. Addition of the polymers led to a clear increase in the relative abundance of nitrogen (due to the acrylamide unit of the polymers) demonstrating the presence of polymers on the nanoparticle surface.

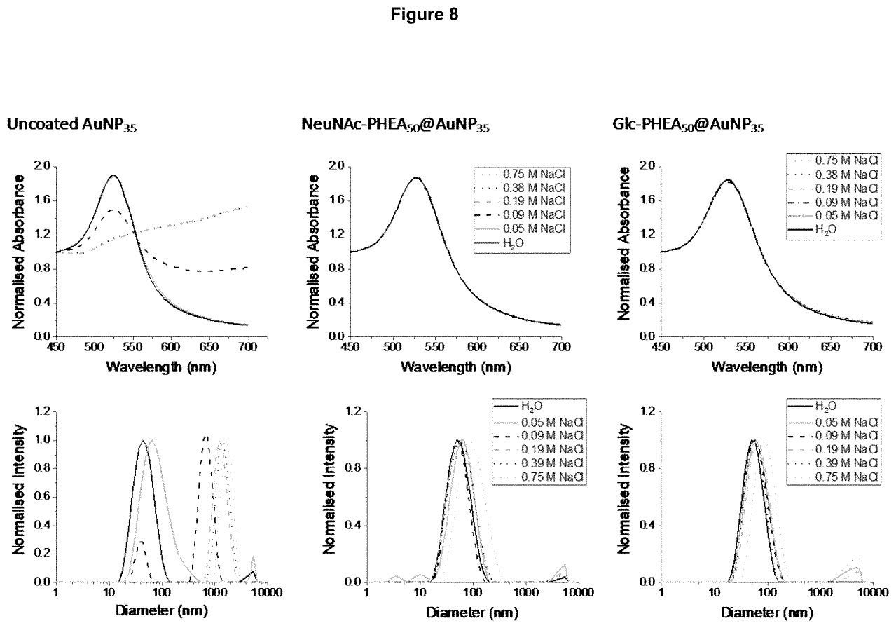

The polymeric tethers (i.e. Linkers) were used both to capture the glycans and to provide colloidal stability to the gold nanoparticles. FIG. 8 shows UV-Visible and dynamic light scattering analysis of nanoparticles with an NaCl gradient. The particles without polymer rapidly aggregated in all saline conditions, but with the polymer coating, the nanoparticles were stable to at least 0.75 M NaCl confirming that the particles are sterically stabilized by multiple polymer chains.

TABLE 3
Elemental composition of nanoparticles determined
by X-ray photoelectron spectroscopy

| | | | Elemental | | | | | |
|---|---|---|---|---|---|---|---|---|
| Particle Composition | | | Percentage Composition (%) | | | | Elemental Ratios | |
| AuNP (nm) | PHEA DP | Sugar | C 1s | O 1s | N 1s | Au 4f | N1s/C1s | N1s/Au4f |
| 16 | 0 | citrate | 55.21 | 43.91 | 0.64 | 0.24 | 0.012 | 2.67 |
| 16 | 40 | NeuNAc | 65.89 | 28.33 | 4.70 | 1.08 | 0.071 | 4.36 |
| 16 | 50 | 2,3SL | 70.44 | 26.45 | 1.47 | 1.63 | 0.021 | 0.90 |
| 16 | 50 | 2,6SL | 67.89 | 29.56 | 1.44 | 1.10 | 0.021 | 1.31 |
| 16 | 50 | NeuNAc | 61.36 | 32.00 | 5.19 | 1.45 | 0.085 | 3.58 |
| 35 | 0 | citrate | 54.59 | 44.17 | 0.88 | 0.37 | 0.016 | 2.39 |
| 35 | 40 | 2,3SL | 67.99 | 29.55 | 1.39 | 1.07 | 0.020 | 1.30 |
| 35 | 40 | 2,6SL | 57.69 | 40.27 | 1.42 | 0.61 | 0.025 | 2.31 |
| 35 | 40 | NeuNAc | 60.69 | 31.49 | 5.70 | 2.12 | 0.094 | 2.68 |
| 35 | 50 | 2,3SL | 68.45 | 28.75 | 1.50 | 1.29 | 0.022 | 1.17 |
| 35 | 50 | 2,6SL | 68.08 | 30.04 | 1.12 | 0.76 | 0.017 | 1.48 |
| 35 | 50 | NeuNAc | 59.49 | 33.16 | 5.66 | 1.70 | 0.095 | 3.32 |

X-Ray Photoelectron Spectroscopy Method

The samples were attached to electrically-conductive carbon tape, mounted on to a sample bar and loaded in to a Kratos Axis Ultra DLD spectrometer which possesses a base pressure below 1×10⁻¹⁰ mbar. XPS measurements were performed in the main analysis chamber, with the sample being illuminated using a monochromated Al Kα x-ray source. The measurements were conducted at room temperature and at a take-off angle of 90° with respect to the surface parallel. The core level spectra were recorded using a pass energy of 20 eV (resolution approx. 0.4 eV), from an analysis area of 300 μm×700 μm. The spectrometer work function and binding energy scale of the spectrometer were calibrated using the Fermi edge and 3d_{5/2} peak recorded from a polycrystalline Ag sample prior to the commencement of the experiments. In order to prevent surface charging the surface was flooded with a beam of low energy electrons throughout the experiment and this necessitated recalibration of the binding energy scale. To achieve this, the C—C/C—H component of the C 1s spectrum was referenced to 285.0 eV. The data were analysed in the CasaXPS package, using Shirley backgrounds and mixed Gaussian-Lorentzian (Voigt) lineshapes. For compositional analysis, the analyser transmission function has been determined using clean metallic foils to determine the detection efficiency across the full binding energy range.

### Example 4: Biolayer Interferometry Analysis of SARS-CoV-2 Spike Protein with Glyconanoparticles

Recombinant S1 subunit of SARS-CoV-2 spike protein was immobilized onto biolayer interferometry (BLI) sensors, and interrogated by the glycoparticles. This replicates a lateral flow situation. We used S1 protein which was expressed in mammalian cells in order to ensure correct glycosylation (and hence potential steric hindrance) was present as in the native protein; this was also confirmed with binding against E. coli-expressed protein.

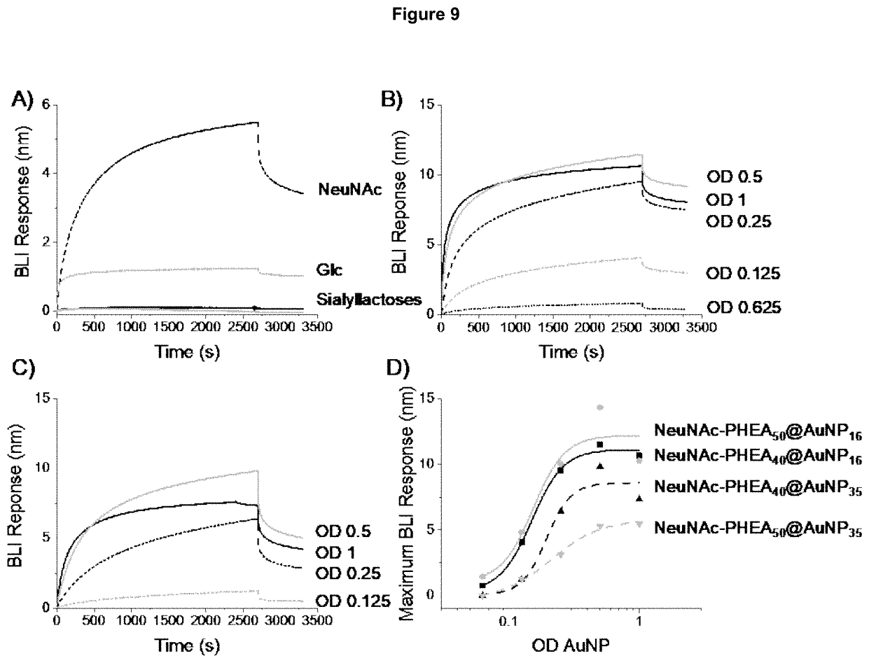

FIG. 9 shows BLI curves of the panel of glycoparticles against the S1 protein of SARS-CoV-2.

FIG. 9A shows that on a nanoparticle scaffold, NeuNAc lead to dramatically more binding compared to either of the sialyllactose isomers (i.e. 2,3-sialyllactose and 2,6-sialyllactose), and against a monosaccharide control (i.e. glucose). X-ray photoelectron spectroscopy analysis of these particles revealed that the monosaccharide-terminated polymers (i.e. NeuNAc and glucose) lead to a higher grafting density than the trisaccharide-terminated polymers (i.e. sialyllactoses) by a ratio of 2 (35 nm) to 3 (16 nm); the difference in glycan size may explain this observation. The strong binding of NeuNAc (FIG. 1C) agrees with the structurally-related MERS spike protein which engages this ligand strongly, and justifies this reductionist approach.

To evaluate the impact of particle size on binding, PHEA₄₀ was used as the tether as it lead to stable colloidal dispersions on both 16 and 35 nm gold (relevant diameters for LFDs); and again used to interrogate SARS-CoV-2, S1 (see FIGS. 9B and 9C).

Dose dependency, as shown in FIG. 9D, showed similar trends for both sizes of particles. (Note, plots are made in terms of OD (at 520 nm)).

Clear and black half are 96-well plates that were purchased from Greiner Bio-one. Streptavidin (SA) biosensors were purchased from Forte Bio. Lectins and hemagglutinins were biotinylated using EZ-Link sulfo-NHS-LC-biotin reagent from Thermo Fisher Scientific using standard procedure (20-fold molar excess of biotin reagent, conjugation performed in PBS buffer and isolated using Amicon Ultra-0.5 mL 3000 MWCO centrifugal filters from Merck Millipore).

### Example 5. Lateral Flow Analysis of Binding

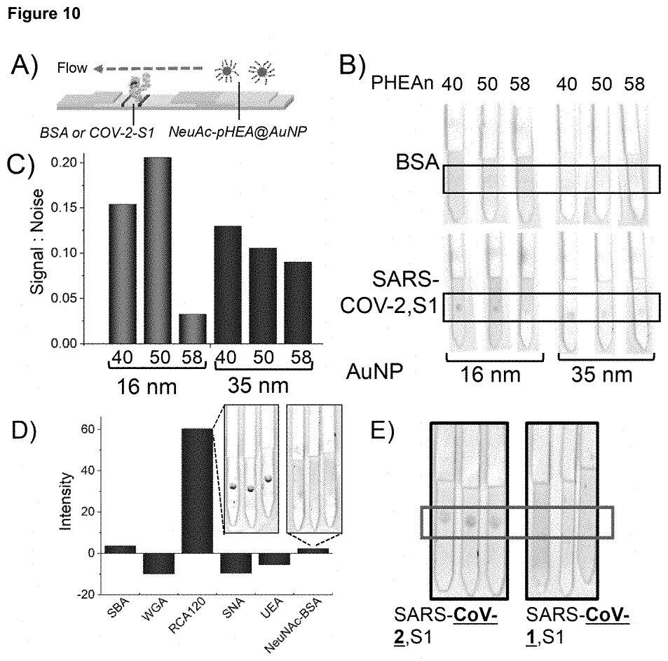

The performance of a lateral flow device depends upon not only the affinity of the capture ligand (in this case N-acetyl neuraminic acid) but also on the flow of the particles. ‘Half’ lateral flow assays (FIG. 10A) were set up to optimize the particles. In this, the test line was either BSA (negative control for non-specific binding) or immobilized SARS-CoV-2, S1; and nanoparticles were ran against them.

16 nm particles gave stronger signals than the 35 nm particles (see FIGS. 10B and 10C), but also more background; hence 35 nm particles were used from this point onwards,

Blocking of the particles with BSA before running was also explored to reduce background. It was found that for the NeuNAc particles blocking was not required (due to the low background), but for the other glycans blocking could reduce background.

Encouraged by these results, the specificity and function of the particles was tested against a panel of immobilized lectins. Total signal intensity is plotted in FIG. 10D confirming the NeuNAc does not show non-specific binding to the wrong lectin. This is a significant benefit of the polymer-stabilization which provides a steric shield. The only lectin which bound was RCA120, which is known to have some affinity towards sialic acids.

To further test specificity in a more challenging situation, the particles were screened against the spike protein of SARS-CoV-2, S1 (i.e. the desired target) and also against the S1 spike domain of a previous zoonotic coronavirus (SARS-CoV-1, which was responsible for 2003 ‘SARS’ outbreak), FIG. 10E. As can clearly be seen, the NeuNAc particle system has clear preference for SARS-CoV-2, highlighting the selectivity of the present system.

Materials

Nitrocellulose Immunopore RP 90-150 s/4 cm 25 mm was purchased from GE Healthcare. Lateral flow backing cards 60 mm by 301.58 mm (KN-PS1060.45 with KN211 adhesive) and lateral flow cassettes (KN-CT105) were purchased from Kenosha Tapes. Cellulose fibre wick material 20 cm by 30 cm by 0.825 mm (290 gsm and 180 ml/min) (Surewick CFSP223000) was purchased from EMD Millipore. Glass fibre conjugate pads (GFCP103000) 10 mm by 300 mm was purchased from Merck. Sample pads Thick Chromatography Paper, Grade 237, Ahlstrom 20 cm by 20 cm were purchased from VWR International.

Protocol for Manufacturing Lateral Flow Strips

Backing cards were cut to size by removal of 20 mm using a guillotine. Nitrocellulose was added to the backing card by attaching the plastic backing of the nitrocellulose to the self-adhesive on the card. The wick material was then added to the backing card so it overlaps with the nitrocellulose by ˜5 mm. The lateral flow strips were cut to size of width 2-3 mm.

Protocol for Test Line Addition to the Lateral Flow Strips

1 μl of the test line solution was added to the test strip using a micropipette fitted with 10 μl tip, the test line was spotted ˜1 cm from the non-wick end of the strip. The strips were dried at 37° C. in an oven for 30 minutes. The tests strips were allowed to cool to room temperature before testing.

Protocol for Running Lateral Flow Test without Target Analyte in Buffer

The running buffer of total volume 50 μl was made as follows; 5 μl AuNPs (OD10), 5 μl lateral flow assay buffer—10×HEPES buffer, 40 μl water. This gives a final buffer of 10 mmol of HEPES, 0.150 mol of NaCl, 0.1 mmol of CaCl2), 0.08% w/v. NaN3, 0.05% w/v. of Tween-20 and 1% w/v. of poly(vinyl pyrrolidone)400. The running solution was then agitated on a roller for 5 minutes. 45 μl of this solution was added to a 0.2 ml PCR tube, standing vertically. In some cases 1% 2/v of poly(vinyl pyrrolidone)400 was used.

Image Analysis of Lateral Flow Strips

Strips were scanned using a Kyocera TASKalfa 5550ci printer to a pdf file that was converted to a jpeg. The jpegs were analysed in Image J 1.51 using the plot profile function to create a data set exported to Microsoft Excel. The data was exported to Origin 2019 64 Bit and trimmed to remove pixel data not from the strip surface. The data was aligned and averaged (mean). The data was then reduced by number of groups to 100 data points (just the nitrocellulose surface) and plotted as Grey value (scale) vs Relative distance along the 100 data points.

Lateral Flow Signal Intensity Analysis

Relative distance pixel 1 to 10 and 51-60 (area around the test line), excluding pixels that contributed to the signal peak were averaged (mean). This average was subtracted from the lowest grey value between 11 to 50.

### Example 6. Further Lateral Flow Analysis of Binding

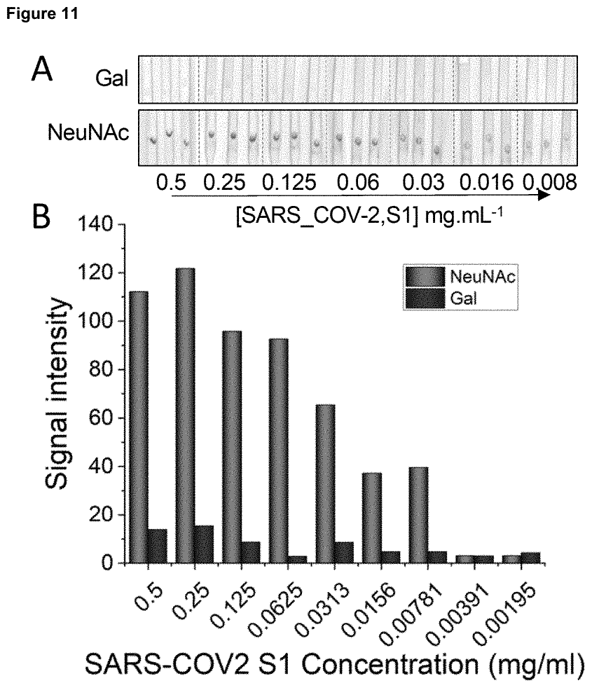

To explore the detection limits and specificity of the nanoparticles, NeuNAc (positive) and galactose (Gal, negative control) nanoparticles were screened against a dilution series of SARS-CoV-2, S1 protein (see FIG. 11) immobilized onto the lateral flow surface. At the very highest concentration (0.5 mg·mL⁻¹), Gal particles showed very weak binding which was far less than NeuNAc, with the latter showing strong binding with an apparent limit of detection being below 8 μg·mL⁻¹ or approximately 8 nM.

### Example 7. Lateral Flow Cassette Assembly

Nitrocellulose was added to the backing card by attaching the plastic backing of the nitrocellulose to the self-adhesive on the card. The wick material was then added to the backing card so it overlapped with the nitrocellulose by ˜5 mm. The strips were then cut to size of width ˜3 mm so they sat in the cassettes without the need for excess force to fit. The conjugate pad was added to the backing card, so it overlapped with the nitrocellulose by ˜3.5 mm.

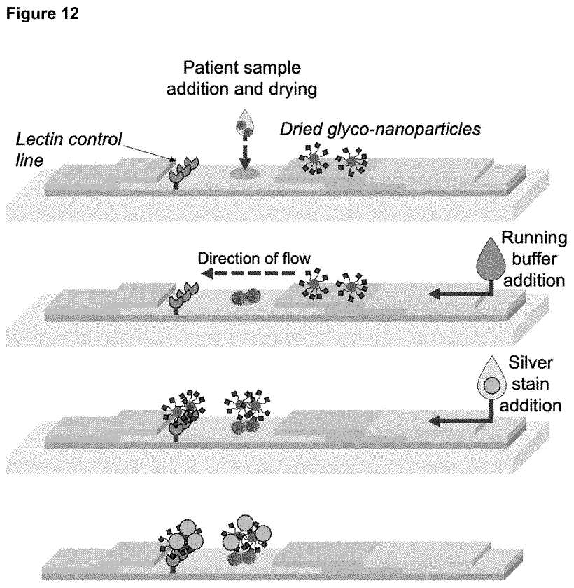

The conjugate pads were made as follows. Strips of the conjugate pad material were agitated for 30 minutes in a solution of 0.1% Tween-20 (blocking solution). The strips were then patted dry and baked overnight at 37° C. in an oven. The conjugate pads were cut to size (3 mm width) and placed individually into the wells of a 384-well microplate. 20 μL 1× conjugate pad buffer solution (1% w/v. of poly(vinyl pyrrolidone)400 (Average Mw˜40,000 g·mol−1), 5% w/v. trehalose, 1% w/v. sucrose and 0.01% w/v. Tween-20) containing OD3 AuNPs was added to the top of each conjugate pad in the wells. The pads were dried overnight at 37° C. in an oven. The completed pads were stored in an airtight box containing desiccant until addition to the strips. Following conjugate pad addition to the strip, the sample pad was cut to size of 20 mm by 6 mm and was added to the backing card, overlapping with the conjugate pad by ˜6.5 mm and straddling the backing card evenly. The completed strip was then added to the cassettes and sealed. A control line of 1 μL of RCA120 (1 mg/mL) was added to the nitrocellulose strip using a micropipette fitted with a 10 μL tip. A control line was added ˜1.5 cm from the non-wick end of the nitrocellulose surface. The strips were dried at 37° C. in an oven for 30 minutes. FIG. 12 exemplifies this and the running of the tests.

### Example 8. Diagnostic Demonstration Using Primary Patient Swabs

Surplus nasal swabs eluates (which had been eluted and heat inactivated as part of clinical investigation of symptomatic patient/staff) and assessed by real-time PCR, were used. To each primary swab sample was added 2000 μL of molecular grade water (if one swab) or 2500 μL of molecular grade water (if two swabs were in a universal container). These were then vortexed and allowed to settle for 5 minutes. All liquid was transferred from the primary container into a 13 mm×75 mm tube. These tubes were heat inactivated at 85° C. for 10 minutes. The specimens were then used for testing with the lateral flow devices.

Test lines were made by direct addition of 2×1 μL of the specimen using a pipette, onto the nitrocellulose strip. The sample was spotted ˜1 cm from the non-wick end of the nitrocellulose surface. The strips were dried at 37° C. in an oven.

Protocol for Running Lateral Flow Tests

100 μL of the running buffer (HEPES containing 2% PVP) was added to the cassette well. The test was run for 15 minutes, before an additional 100 μL of running buffer was then added and after a further 15 minutes photos were taken. A silver stain (silver enhance kit from Aldrich) was then added to the cassette well (100 μL) and run for 20 minutes, after this time photos were then taken.

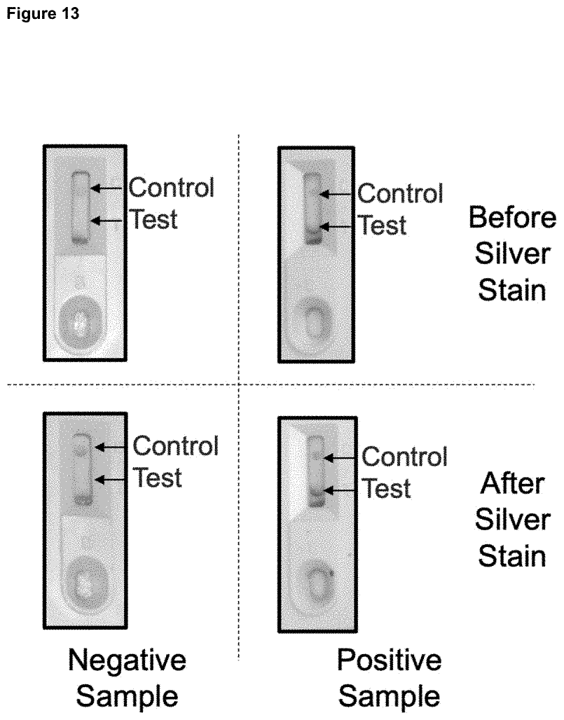

The results are shown in FIG. 13. A positive sample had a visible test line and a control line which was visible either after first run (or after the silver staining). A negative sample had no test line visible. Failed devices where no control line or the sample did not run were excluded from analysis.

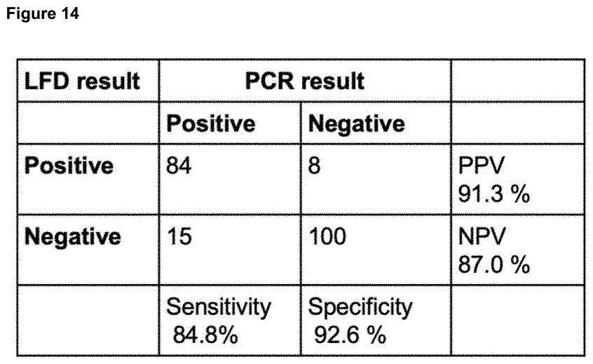

The following was used to determine performance:
Sensitivity=TP/(TP+FN);
Specificity=TN/(TN+FP);
PPV=TP/(TP+FP);
NPV=TN/(TN+FN)
   where TP=true positive; TN=true negative; FN=false negative; and FP=false positive. Performance data is shown in FIG. 14. Performance without silver staining was: sensitivity=67.7%, Specificity=96.3%. Performance after silver staining was: sensitivity 84.8%, Specificity 92.6%.

### Example 9. Application to Other Sialic-Acid Binding Viruses

Specific linker-sialic acid combinations may also be used to specifically detect viruses other than coronaviruses. Lateral flow cassettes as described in Example 7 were used. 0.5 mg/mL of the hemagglutinin was added to the lateral flow cassettes as a test line (drying for 10 minutes at 37° C.) and 100 μL buffer was run for 20 minutes and photos taken. The hemagglutinins used were:

H7 Hemagglutinin (HA) Protein from Influenza Virus, A/Canada/rv444/2004 (H7N3), Recombinant from Baculovirus, NR-43740, NIAID, NIH;
H7 Hemagglutinin (HA) Protein from Influenza Virus, A/Shanghai/1/2013 (H7N9), Recombinant from Baculovirus, NR-44079, NIAID, NIH;
H3 Hemagglutinin (HA) Protein from Influenza Virus, A/New York/55/2004 (H3N2), Recombinant from Baculovirus, NR-19241 and NIAID, NIH; and
H1 Hemagglutinin (HA) Protein with C-Terminal Histidine Tag from Influenza Virus, A/Brisbane/59/2007 (H1N1),

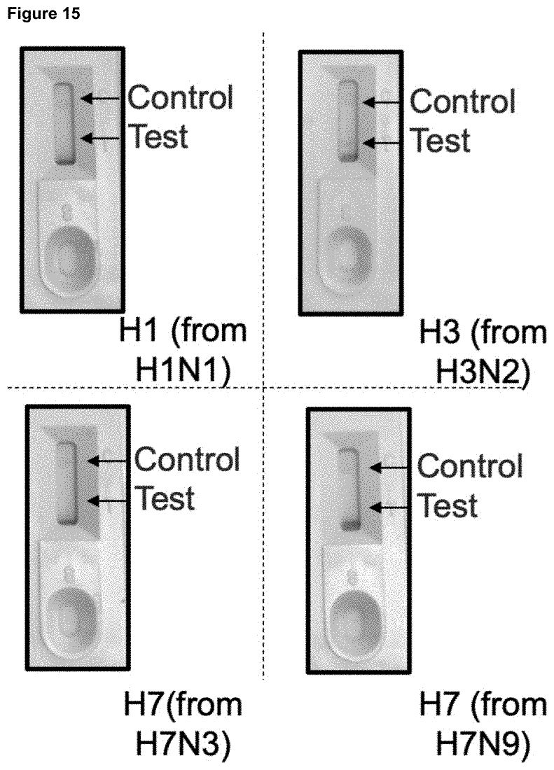

The results are shown in FIG. 15. The results show that influenza H3 showed binding in the lateral flow cassette.

### Example 10: Detection of SARS-COV-2 Variant Spike Protein

In order to establish whether such devices were capable of detecting new variants of SARS-COV-2, a number of truncated recombinant spike proteins containing mutations associated with SARS-COV-2 variants were produced (in E. coli). The primary amino acid sequence is given in SEQ ID NO: 10 The mutations which were tested are indicated below.

| First detection | PANGO | |
|---|---|---|
| location | Lineage | Relevant mutations |
| Denmark | Not registered | H69-V70 deletion |
| United Kingdom | B.1.1.7 | H69-V70 deletion, Y144 deletion |
| South Africa | B.1.351 | L18F, D80A, D215G, R246I |

Test lines were made by direct addition of 1 μL of 5 μM of the variant spike protein in PBS using a pipette, onto the nitrocellulose strip of an assembled device. The strips were dried at 37° C. in an oven.

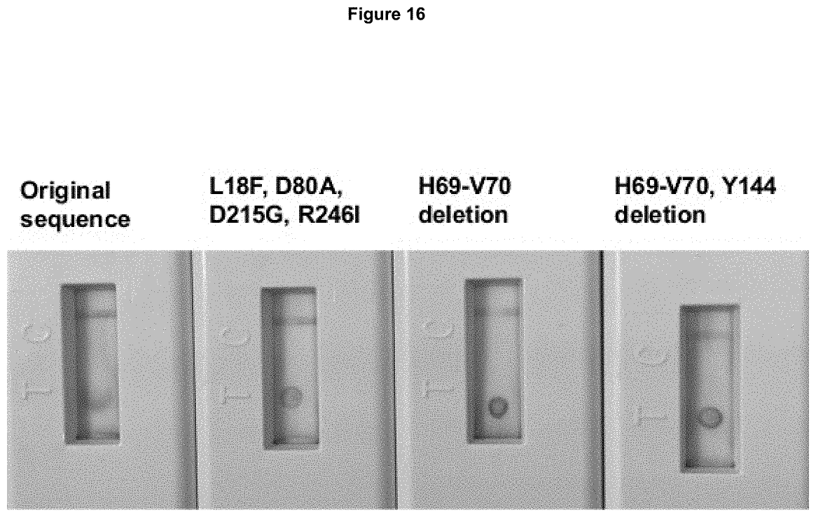

The tests were performed using the buffers as described in Example 8. The results are shown in FIG. 16. These results show that such devices of the invention were capable of detecting the Denmark, UK and South African variants of SARS-COV-2.

### REFERENCES

(1) Zhou, P.; Yang, X.-L.; Wang, X.-G.; Hu, B.; Zhang, L.; Zhang, W.; Si, H.-R.; Zhu, Y.; Li, B.; Huang, C.-L.; et al. A Pneumonia Outbreak Associated with a New Coronavirus of Probable Bat Origin. Nature 2020, 579 (7798), 270-273.
(2) Xie, X.; Zhong, Z.; Zhao, W.; Zheng, C.; Wang, F.; Liu, J. Chest CT for Typical 2019-NCoV Pneumonia: Relationship to Negative RT-PCR Testing. Radiology 2020, 200343.
(3) Li, Y.; Yao, L.; Li, J.; Chen, L.; Song, Y.; Cai, Z.; Yang, C. Stability Issues of RT-PCR Testing of SARS-CoV-2 for Hospitalized Patients Clinically Diagnosed with COVID-19. J. Med. Virol 2020, 1-6.
(4) Huang, P.; Liu, T.; Huang, L.; Liu, H.; Lei, M.; Xu, W.; Hu, X.; Chen, J.; Liu, B. Use of Chest CT in Combination with Negative RT-PCR Assay for the 2019 Novel Coronavirus but High Clinical Suspicion. Radiology 2020, No. PG-200330-200330, 200330.
(5) Hase, R.; Kurita, T.; Muranaka, E.; Sasazawa, H.; Mito, H.; Yano, Y. A Case of Imported COVID-19 Diagnosed by PCR-Positive Lower Respiratory Specimen but with PCR-Negative Throat Swabs. Infect. Dis. (Auckl). 2020.
(6) Crane, M. M.; Organon MV. Diagnostic Test Device—U.S. Pat. No. 3,579,306A, January 1969.
(7) Ezennia, I. J.; Nduka, S. O.; Ekwunife, O. I. Cost Benefit Analysis of Malaria Rapid Diagnostic Test: The Perspective of Nigerian Community Pharmacists. Malar. J. 2017, 16 (1), 7-16.
(8) Tawiah, T.; Hansen, K. S.; Baiden, F.; Bruce, J.; Tivura, M.; Delimini, R.; Amengo-Etego, S.; Chandramohan, D.; Owusu-Agyei, S.; Webster, J. Cost-Effectiveness Analysis of Test-Based versus Presumptive Treatment of Uncomplicated Malaria in Children under Five Years in an Area of High Transmission in Central Ghana. PLoS One 2016, 11 (10), e0164055.
(9) Phan, J. C.; Pettitt, J.; George, J. S.; Fakoli, L. S.; Taweh, F. M.; Bateman, S. L.; Bennett, R. S.; Norris, S. L.; Spinnler, D. A.; Pimentel, G.; et al. Lateral Flow Immunoassays for Ebola Virus Disease Detection in Liberia. J. Infect. Dis. 2016, 214 (suppl 3), S222-S228.
(10) Mao, X.; Ma, Y.; Zhang, A.; Zhang, L.; Zeng, L.; Liu, G. Disposable Nucleic Acid Biosensors Based on Gold Nanoparticle Probes and Lateral Flow Strip. Anal. Chem. 2009, 81 (4), 1660-1668.
(11) Damborský, P.; Koczula, K. M.; Gallotta, A.; Katrlík, J. Lectin-Based Lateral Flow Assay: Proof-of-Concept. Analyst 2016, 141 (23), 6444-6448.
(12) Connor, R. J.; Kawaoka, Y.; Webster, R. G.; Paulson, J. C. Receptor Specificity in Human, Avian, and Equine H2 and H3 Influenza Virus Isolates. Virology 1994, 205 (1), 17-23.
(13) Marín, M. J.; Rashid, A.; Rejzek, M.; Fairhurst, S. A.; Wharton, S. A.; Martin, S. R.; McCauley, J. W.; Wileman, T.; Field, R. A.; Russell, D. A. Glyconanoparticles for the Plasmonic Detection and Discrimination between Human and Avian Influenza Virus. Org. Biomol. Chem. 2013, 11 (41), 7101.
(14) Richards, S.-J.; Baker, A. N.; Walker, M.; Gibson, M. I. Polymer-Stabilized Sialylated Nanoparticles: Synthesis, Optimization, and Differential Binding to Influenza Hemagglutinins. Biomacromolecules 2020, 21 (4), 1604-1612.
(15) Qing, E.; Hantak, M.; Perlman, S.; Gallagher, T. Distinct Roles for Sialoside and Protein Receptors in Coronavirus Infection. MBio 2020, 11 (1).
(16) Hulswit, R. J. G.; Lang, Y.; Bakkers, M. J. G.; Li, W.; Li, Z.; Schouten, A.; Ophorst, B.; Van Kuppeveld, F. J. M.; Boons, G. J.; Bosch, B. J.; et al. Human Coronaviruses 0C43 and HKU1 Bind to 9-O-Acetylated Sialic Acids via a Conserved Receptor-Binding Site in Spike Protein Domain A. Proc. Natl. Acad. Sci. U.S.A 2019, 116 (7), 2681-2690.
(17) Huang, X.; Dong, W.; Milewska, A.; Golda, A.; Qi, Y.; Zhu, Q. K.; Marasco, W. A.; Baric, R. S.; Sims, A. C.; Pyrc, K.; et al. Human Coronavirus HKU1 Spike Protein Uses O-Acetylated Sialic Acid as an Attachment Receptor Determinant and Employs Hemagglutinin-Esterase Protein as a Receptor-Destroying Enzyme. J. Virol. 2015, 89 (14), 7202-7213.
(18) Li, W.; Hulswit, R. J. G.; Widjaja, I.; Raj, V. S.; McBride, R.; Peng, W.; Widagdo, W.; Tortorici, M. A.; Van Dieren, B.; Lang, Y.; et al. Identification of Sialic Acid-Binding Function for the Middle East Respiratory Syndrome Coronavirus Spike Glycoprotein. Proc. Natl. Acad. Sci. U.S.A 2017, 114 (40), E8508-E8517.
(19) Georgiou, P. G.; Baker, A. N.; Richards, S. J.; Laezza, A.; Walker, M.; Gibson, M. I. Tuning Aggregative versus Non-Aggregative Lectin Binding with Glycosylated Nanoparticles by the Nature of the Polymer Ligand. J. Mater. Chem. B 2020, 8 (1), 136-145.
(20) Richards, S.-J.; Gibson, M. I. Optimization of the Polymer Coating for Glycosylated Gold Nanoparticle Biosensors to Ensure Stability and Rapid Optical Readouts. ACS Macro Lett. 2014, 3 (10), 1004-1008.
(21) Haiss, W.; Thanh, N. T. K.; Aveyard, J.; Fernig, D. G. Determination of Size and Concentration of Gold Nanoparticles from UV—Vis Spectra. Anal. Chem. 2007, 79 (11), 4215-4221.
(22) Hulswit, R. J. G.; Lang, Y.; Bakkers, M. J. G.; Li, W.; Li, Z.; Schouten, A.; Ophorst, B.; van Kuppeveld, F. J. M.; Boons, G.-J.; Bosch, B.-J.; Huizinga, E. G.; de Groot, R. J. Human Coronaviruses OC43 and HKU1 Bind to 9-O-Acetylated Sialic Acids via a Conserved Receptor-Binding Site in Spike Protein Domain A. Proc. Natl. Acad. Sci. 2019,116 (7), 2681-2690.
(23) Tortorici, M. A.; Walls, A. C.; Lang, Y.; Wang, C.; Li, Z.; Koerhuis, D.; Boons, G.-J.; Bosch, B.-J.; Rey, F. A.; de Groot, R. J.; Veesler, D. Structural Basis for Human Coronavirus Attachment to Sialic Acid Receptors. Nat. Struct. Mol. Biol. 2019, 26 (6), 481-489.
(24) Li, W.; Hulswit, R. J. G.; Widjaja, I.; Raj, V. S.; McBride, R.; Peng, W.; Widagdo, W.; Tortorici, M. A.; van Dieren, B.; Lang, Y.; van Lent, J. W. M.; Paulson, J. C.; de Haan, C. A. M.; de Groot, R. J.; van Kuppeveld, F. J. M.; Haagmans, B. L.; Bosch, B.-J. Identification of Sialic Acid-Binding Function for the Middle East Respiratory Syndrome Coronavirus Spike Glycoprotein. Proc. Natl. Acad. Sci. 2017, 114 (40), E8508-E8517.
(25) Burmeister, W. P.; Guilligay, D.; Cusack, S.; Wadell, G.; Arnberg, N. Crystal Structure of Species D Adenovirus Fiber Knobs and Their Sialic Acid Binding Sites. J. Virol. 2004, 78 (14), 7727-7736.
(26) Arnberg, N.; Kidd, A. H.; Edlund, K.; Nilsson, J.; Pring-Åkerblom, P.; Wadell, G. Adenovirus Type 37 Binds to Cell Surface Sialic Acid through a Charge-Dependent Interaction. Virology 2002.
(27) Childs, R. A.; Palma, A. S.; Wharton, S.; Matrosovich, T.; Liu, Y.; Chai, W.; Campanero-Rhodes, M. A.; Zhang, Y.; Eickmann, M.; Kiso, M.; Hay, A.; Matrosovich, M.; Feizi, T. Receptor-Binding Specificity of Pandemic Influenza A (H1N1) 2009 Virus Determined by Carbohydrate Microarray. Nat. Biotechnol. 2009, 27 (9), 797-799.
(28) Kumari, K.; Gulati, S.; Smith, D. F.; Gulati, U.; Cummings, R. D.; Air, G. M. Receptor Binding Specificity of Recent Human H3N2 Influenza Viruses. Virol. J. 2007, 4 (1), 42.
(29) Kubota, M.; Takeuchi, K.; Watanabe, S.; Ohno, S.; Matsuoka, R.; Kohda, D.; Nakakita, S.; Hiramatsu, H.; Suzuki, Y.; Nakayama, T.; Terada, T.; Shimizu, K.; Shimizu, N.; Shiroishi, M.; Yanagi, Y.; Hashiguchi, T. Trisaccharide Containing A2,3-Linked Sialic Acid Is a Receptor for Mumps Virus. Proc. Natl. Acad. Sci. 2016, 113 (41), 11579-11584.
(30) Suzuki, T.; Portner, A.; Scroggs, R. A.; Uchikawa, M.; Koyama, N.; Matsuo, K.; Suzuki, Y.; Takimoto, T. Receptor Specificities of Human Respiroviruses. J. Virol. 2001, 75 (10), 4604-4613.
(31) Rydell, G. E.; Nilsson, J.; Rodriguez-Diaz, J.; Ruvoën-Clouet, N.; Svensson, L.; Le Pendu, J.; Larson, G. Human Noroviruses Recognize Sialyl Lewis x Neoglycoprotein. Glycobiology 2009, 19 (3), 309-320.

ADDITIONAL SEQUENCES
SEQ ID NO: 10
Primary sequence of truncated
SARS-COV-2 (Lineage A)
MFVFLVLLPLVSSQCVNLTTRTQLPPAYTNSF
TRGVYYPDKVFRSSVLHSTQDLFLPFFSNVTW
FHAIHVSGTNGTKRFDNPVLPFNDGVYFASTE
KSNIIRGWIFGTTLDSKTQSLLIVNNATNVVI
KVCEFQFCNDPFLGVYYHKNNKSWMESEFRVY
SSANNCTFEYVSQPFLMDLEGKQGNFKNLREF
VFKNIDGYFKIYSKHTPINLVRDLPQGFSALE
PLVDLPIGINITRFQTLLALHRSYLTPGDSSS
GWTAGAAAYYVGYLQPRTFLLKYNENGTITDA
VDCALDPLSETK
SEQUENCE LISTING FREE TEXT
<210> 10
<223> Primary sequence of truncated
SARS-COV-2 (Lineage A)

1-3. (canceled)

4. A flow device or, a lateral flow device, for detecting the presence of an analyte in a test sample, the device comprising a conjugate zone and a detection zone, wherein:
(a) the conjugate zone comprises a first specific binding partner for the analyte, wherein the first specific binding partner comprises a sialic acid linked to a detectable label,
wherein the first specific binding partner which is linked to a detectable label has the structure:
(i) first specific binding partner-linker-detectable label, or
(ii) first specific binding partner-linker-polymer-detectable label,
wherein the first specific binding partner-linker has one of the following structures: 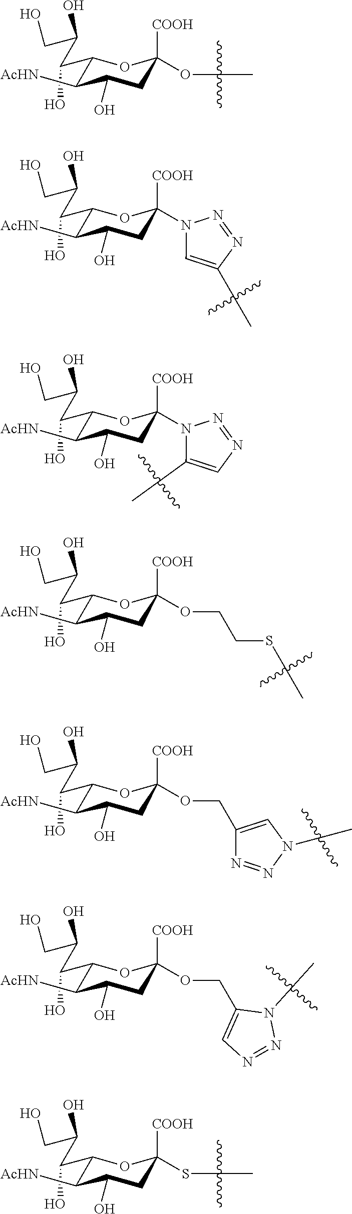 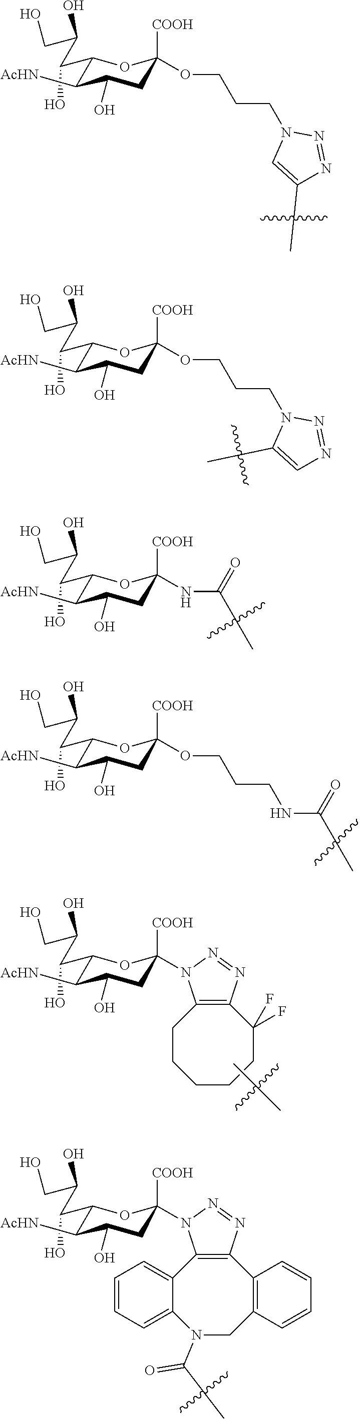 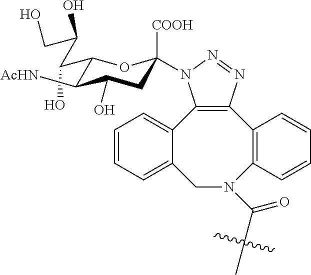
and wherein the first specific binding partner is not immobilized in the conjugate zone; and
(b) the detection zone comprises a zone which is adapted to receive the test sample.

5. The flow device or the lateral flow device as claimed in claim 4, wherein the flow device or the lateral flow device, comprises:
(a) a fluid receiving zone, to which an aqueous solution is applied or is capable of being applied;
(b) a conjugate zone, wherein the first specific binding partner is linked to the detectable label, and wherein the first specific binding partner is not immobilized;
(c) a detection zone, to which the test sample is applied or is capable of being applied;
and optionally one or both of:
(d) a control zone, and
(e) an absorbent zone,
wherein the above zones, when present, are joined in fluid communication, in the above-mentioned order.

6-8. (canceled)

9. The flow device or the lateral flow device as claimed in claim 4, wherein the analyte is a virus particle, coronavirus particle or a coronavirus spike protein.

10. The flow device or the lateral flow device as claimed in claim 4, wherein the analyte is a virus particle or virus protein, and the virus is selected from the group consisting of adenoviruses, influenza viruses, mumps viruses, parainfluenza viruses and noroviruses.

11. The flow device or the lateral flow device as claimed in claim 9, wherein the coronavirus particle is a SARS-CoV-2 particle or the coronavirus spike protein is SARS-CoV-2 S1.

12. The flow device or the lateral flow device as claimed in claim 4, wherein the test sample comprises a nasal swab or throat swab from a subject or sputum from a subject.

13-20. (canceled)

21. The flow device or the lateral flow device as claimed in claim 4, wherein the polymer has one of the following structures: 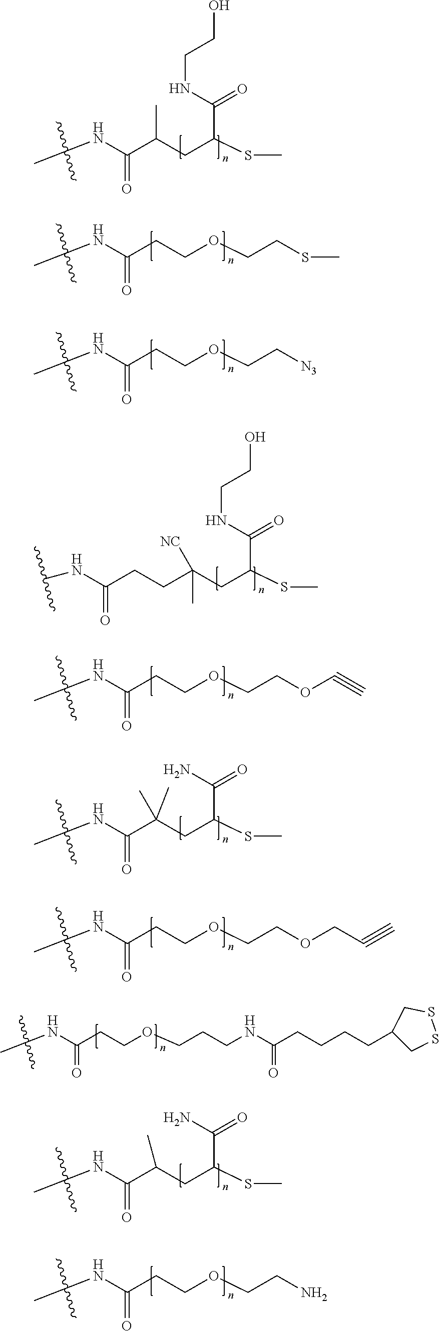 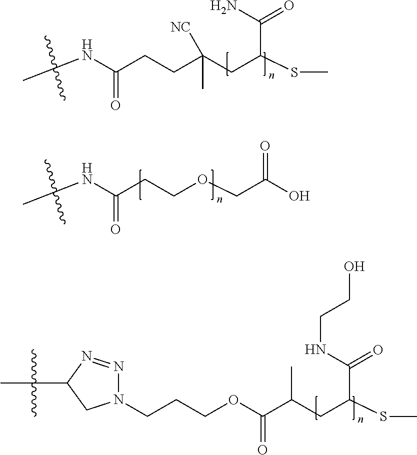
wherein n=1-200,
or wherein the polymer has the structure: 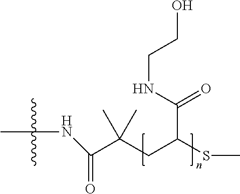
wherein n=40-60.

22. The flow device or the lateral flow device as claimed in claim 21, wherein a plurality of first specific binding partners are linked via polymers to each detectable label, or wherein 500-3000 first specific binding partners are linked via polymers or via linker-polymers to each detectable label.

23. The flow device or the lateral flow device as claimed in claim 4, wherein the detectable label is a nanoparticle, or a gold nanoparticle.

24. The flow device or the lateral flow device as claimed in claim 4, wherein first specific binding partner-detectable label has one of the following structures: 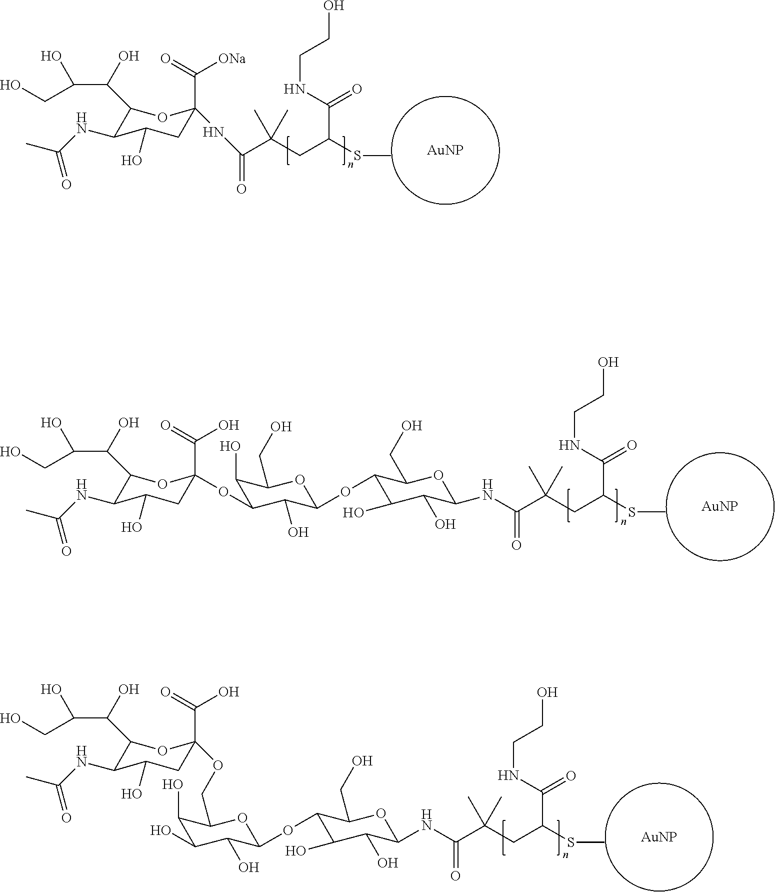
wherein n=30-70, or 40-60, or about 48, 50 or 58;
and wherein AuNP represents a gold nanoparticle, and tautomers, enantiomers and diastereomers thereof.

25-32. (canceled)
